(19) United States
(12) Patent Application Publication
(10) Pub. No.: US 2004/0014722 A1
(43) Pub. Date: Jan. 22, 2004

(54) Nucleosides, preparation thereof and use as inhibitors of RNA viral polymerases

(75) Inventors: Yarlagadda S. Babu, Birmingham, AL (US); Pooran Chand, Birmingham, AL (US); Yahya El-Kattan, Hoover, AL (US); Minwan Wu, Vestavia Hills, AL (US)

Correspondence Address: CONNOLLY BOVE LODGE & HUTZ LLP, SUITE 800, 1990 M STREET NW, WASHINGTON, DC, 20036-3425, US

(73) Assignee: BIOCRYST PHARMACEUTICALS, INC.

(21) Appl. No.: 10/437,179

(22) Filed: May 14, 2003

Related U.S. Application Data
(63) Continuation-in-part of application No. PCT/US02/36621, filed on Nov. 14, 2002.
(60) Provisional application No. 60/331,322, filed on Nov. 14, 2001.

(51) Int. Cl. A61K 31/675; C07F 9/6512

(52) U.S. Cl. 514/81; 544/243; 544/244; 514/86

(57) ABSTRACT

Compounds represented by the formula:

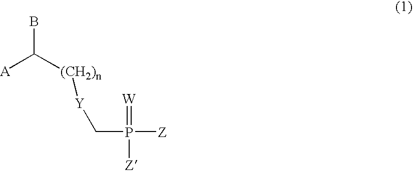

Wherein A is —(CH₂)ₙ—R, —CH═CH₂, —CH₂—CH═CH₂, —O—(CH₂)ₙ—R, —CH(OH)CH₃, —CH(OH)—CH₂OH, —CH₂—CH(OH)CH₃, —CH₂—CH(OH)—CH₂OH, —CH(OH)—CH(OH)—CH₃;

R is H, OH, F, N₃, NH₂, CO₂H, SH, alkyl, S-alkyl, O-acyl, CONH₂, CONH-alkyl;

Z and Z′ independently is OR¹, OR², O—(CH₂)ₙ—O-alkyl or aminoacids and esters thereof;

R¹ and R² independently is H, alkyl, aryl, pivaloyloxymethyl, phthalyl or substituted phthalyl, C(R³)₂OC(O)X(R⁴)a,

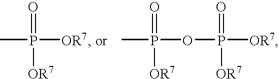

R³ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, or —OR⁵;

R⁴ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, —N(R⁶)₂ or —OR⁵;

R⁵ is C₁-C₁₂ alkyl, C₂-C₁₂ alkenyl, C₂-C₁₂ alkynyl or C₅-C₁₂ aryl;

provided that at least one R⁴ is not H; and a is 1 when X is CH₂, or direct bond, or 1 or 2 when X is N with the proviso that when a is 2 and X is N, (a) two N-linked R groups can be taken together to form a carbocyclic or oxygen containing heterocycle, (b) one N-linked R⁴ additionally can be —OR⁵ or (c) both N-linked R⁴ groups can be —H;

R⁶ is H or C₁-C₈ alkyl;

R⁷ is selected from H, alkyl, alkenyl, alkynyl, aryl, acyloxyalkyl, and pivaloyloxyalkyl;

n is 1-3;

Y is O, S or NH;

W is O or S;

B is selected from the group consisting of adenine, guanine, cytosine, uracil, thymine, modified purines, pyrimidines and pyridines are provided.

### TECHNICAL FIELD

The present invention relates to certain nucleosides and particularly to nucleosides that are useful as inhibitors of viral RNA polymerases such as, but not limited to, hepatitis B, hepatitis C, Polio, Coxsackie A and B, Rhino, Echo, small pox, Ebola, and West Nile virus polymerases.

The present invention also relates to pharmaceutical compositions comprising the compounds of the present invention, as well as methods of using the compounds in inhibiting viral RNA polymerases and treating patients suffering from diseases caused by various RNA viruses.

The present invention also relates to a method for producing the compounds of the present invention.

### BACKGROUND OF THE INVENTION

Hepatitis C virus (HCV), as a particular example of an RNA virus, has infected an estimated 170 million people worldwide, leading to a major health crisis as a result of the disease. Indeed, during the next few years the number of deaths from HCV-related liver disease and hepatocellular carcinoma may overtake those caused by AIDS. Egypt is the hardest hit country in the world, with 23% of the population estimated to be carrying the virus; whereas, in the USA the prevalence of chronic infections has recently been determined to be around 1.87% (2.7 million persons). HCV infections become chronic in about 50% of cases. Of these, about 20% develop liver cirrhosis that can lead to liver failure, including hepatocellular carcinoma.

The NS5B region of HCV encodes a 65 KDa RNA-dependent RNA polymerase (RdRp) thought to be responsible for viral genome replication. RdRps function as the catalytic subunit of the viral replicase required for the replication of all positive-strand viruses. The NS5B protein has been well characterized, shown to possess the conserved GDD motif of RNA-dependent RNA polymerases and in vitro assay systems have been reported. Cellular localization studies revealed that NS5B is membrane-associated in the endoplasmic reticulum like NS5A, suggesting that those two proteins may remain associated with one another after proteolytic processing. Additional evidence suggests that NS3, NS4A and NS5B interact with each other to form a complex that function as part of the replication machinery of HCV.

The X-ray crystal structure of NS5B apoenzyme has now been determined and three very recent publications describe the unusual shape of the molecule. This unique shape for a polymerase, resembling a flat sphere, is attributed to extensive interactions between the fingers and thumb subdomains in such a way that the active site is completely encircled, forming a cavity 15 Å across and 20 Å deep. Modeling studies showed that the NS5B apoenzyme can accommodate the template-primer without large movement of the subdomains, suggesting that the structure is preserved during the polymerization reaction.

There are only a few reports of weak inhibitors of the polymerase. These include some nucleotide analogues, gliotoxin and the natural product cerulenin.

Accordingly, it would be desirable to develop inhibitors of RNA viral polymerases.

### SUMMARY OF THE INVENTION

The present invention relates to novel compounds and in particular, compounds that are represented by the formula:

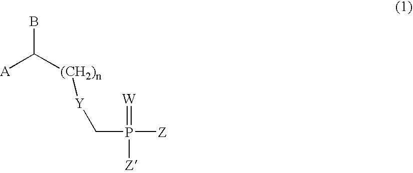

Wherein A is —(CH₂)ₙ—R, —CH═CH₂, —CH₂—CH═CH₂, —O—(CH₂)ₙ—R, —CH(OH)CH₃, —CH(OH)—CH₂OH, —CH₂—CH(OH)CH₃, —CH₂—CH(OH)—CH₂OH, —CH(OH)—CH(OH)—CH₃;

R is H, OH, F, N₃, NH₂, CO₂H, SH, alkyl, S-alkyl, O-acyl, CONH₂, CONH-alkyl;

Z and Z′ independently is OR¹, OR², O—(CH₂)ₙ—O-alkyl or aminoacids and esters thereof;

R¹ and R² independently is H, alkyl, aryl, pivaloyloxymethyl, phthalyl or substituted phthalyl, C(R³)₂OC(O)X(R⁴)a,

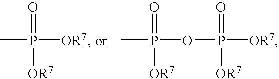

R³ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, or —OR⁵;

R⁴ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₂ alkynylaryl, or C₆-C₁₂ alkaryl, any of which is unsubstituted or is substituted with 1 or 2 halo, cyano, azido, nitro, —N(R⁶)₂ or —OR⁵;

R⁵ is C₁-C₁₂ alkyl, C₂-C₁₂ alkenyl, C₂-C₁₂ alkynyl or C₅-C₁₂ aryl;

provided that at least one R⁴ is not H; and a is 1 when X is CH₂, or direct bond, or 1 or 2 when X is N with the proviso that when a is 2 and X is N, (a) two N-linked R groups can be taken together to form a carbocyclic or oxygen containing heterocycle, (b) one N-linked R⁴ additionally can be —OR⁵ or (c) both N-linked R⁴ groups can be —H;

R⁶ is H or C₁-C₈ alkyl;

R⁷ is selected from H, alkyl, alkenyl, alkynyl, aryl, acyloxyalkyl, and pivaloyloxyalkyl;

n is 1-3;

Y is O, S or NH;

W is O or S;

B is selected from the group consisting of adenine, guanine, cytosine, uracil, thymine, modified purines and pyrimidines such as inosin-9-yl, 2-amino-purin-9-yl, 2-amino-6-chloro-purin-9-yl, 2-6-diamino-purin-9-yl, 3-carboxamido-1,2,4-triazol-1-yl, 3-deaza-adenin-9-yl, 3-deaza-guanin-9-yl, 3-deaza-inosin-9-yl, 3-deaza-2-amino-purin-9-yl, 3-deaza-2-amino-6-chloro-purin-9-yl, 3-deaza-2,6-diamino-purin-9-yl, 7-deaza-adenin-9-yl, 7-deaza-guanin-9-yl, 7-deaza-inosin-9-yl, 7-deaza-2-amino-purin-9-yl, 7-deaza-2-amino-6-chloro-purin-9-yl, 7-deaza-2-6-diamino-purin-9-yl, 7-deaza-8-aza-adenin-9-yl, 7-deaza-8-aza-guanin-9-yl, 7-deaza-8-aza-inosin-9-yl, 7-deaza-8-aza-2-amino-purin-9-yl, 7-deaza-8-aza-2-amino-6-chloro-purin-9-yl, 7-deaza-8-aza-2-6-diamino-purin-9-yl, 8-aza-adenin-9-yl, 8-aza-guanin-9-yl, 8-aza-inosin-9-yl, 8-aza-2-amino-purin-9-yl, 8-aza-2-amino-6-chloro-purin-9-yl, 8-aza-2-6-diamino-purin-9-yl, 6-thio-purin, 6-methylthiopurin, 5-aza-thymin-1-yl, 5-aza-cytosin-1-yl, 5-aza-uracil-1-yl, 6-aza-thymin-1-yl, 6-aza-cytosin-1-yl, 6-aza-uracil-1-yl, 2-thiouracil-1-yl, 4-thiouracil-1-yl, 2-thiocytosine-1-yl, uracil-5-yl, 2-thiouracil-5-yl, 4-thiouracil-5-yl, substituted pyridine derivatives such as 6-azauracil, and azacytosine. In general, attachment may be at different positions in the ring at nitrogen or carbon. These B ring systems may be substituted with halo, alkyl, substituted alkyl (F, Cl, Br, I, OH), NH₂, N₃, aryl, substituted aryl (F. Cl, Br, I, OH, NH₂), aralkyl.

To further illustrate, some of the purine derivatives, the following structure is taken as B

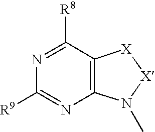

X and X′ is independently CH, N;

R⁸ and R⁹ independately is H, NH₂, OH, SH, Cl, Br, I, aryl, heterocycle, alkyl, alkene, alkyne, S-alkyl, S-aryl, S(O)-alkyl, SO₂-alkyl, SO₂NH₂, SO₂NH-alkyl, SO₂NH-aryl, NH-alkyl, NH-aryl, N(alkyl)₂, N(aryl)₂, O-alkyl, O-aryl, O-heterocycle,

and pharmaceutically acceptable salts thereof and prodrugs thereof.

Exceptions to the above compounds are when A is CH₂OH, CH₂F, CH₂N₃, CH₃ or —CH═CH₂ then B cannot be adenin-9-yl, 1-deazaadenin-9-yl, 7-deaza-8-azaadenin-9-yl, 8-azaadenin-9-yl, guanin-9-yl, 2,6-diaminopurin-9-yl, 2-aminopurin-9-yl, thymin-1-yl, cytosine-1-yl, 5-fluorocytosin-1-yl, 6-azacytosin-1 yl, 5-methylcytosin-1-yl, 5-bromovinyluracil-1-yl, 5-fluorouracil-1-yl or 5-trifluoromethyluracil-1-yl.

2) Compounds represented by the formula:

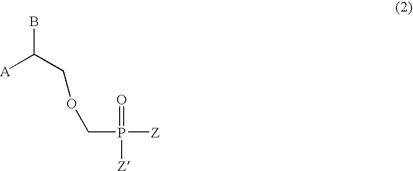

Wherein A is —(CH₂)ₙ—R, —CH═CH₂, —CH₂—CH═CH₂, —O—(CH₂)ₙ—R, —CH(OH)CH₃, —CH(OH)—CH₂OH, —CH₂—CH(OH)CH₃, —CH₂—CH(OH)—CH₂OH, —CH(OH)—CH(OH)—CH₃;

R is H, OH, F, N₃, NH₂, CO₂H, SH, alkyl, S-alkyl, O-acyl, CONH₂, CONH-alkyl;

Z and Z′ independently is OR¹, OR², O—(CH₂)ₙ—O-alkyl or aminoacids and esters thereof;

R¹ and R² independently is H, alkyl, aryl, pivaloyloxymethyl, phthalyl or substituted phthalyl, C(R³)₂OC(O)X(R⁴)a,

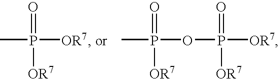

R³ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, or —OR⁵;

R⁴ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, —N(R⁶)₂ or —OR⁵;

R⁵ is C₁-C₁₂ alkyl, C₂-C₁₂ alkenyl, C₂-C₁₂ alkynyl or C₅-C₁₂ aryl;

provided that at least one R⁴ is not H; and a is 1 when X is CH₂, or direct bond, or 1 or 2 when X is N with the proviso that when a is 2 and X is N, (a) two N-linked R groups can be taken together to form a carbocyclic or oxygen containing heterocycle, (b) one N-linked R⁴ additionally can be —OR⁵ or (c) both N-linked R⁴ groups can be —H;

R⁶ is H or C₁-C₈ alkyl;

R⁷ is selected from H, alkyl, alkenyl, alkynyl, aryl, acyloxyalkyl, and pivaloyloxyalkyl;

B is selected from the group consisting of adenine, guanine, cytosine, uracil, thymine, modified purines and pyrimidines such as inosin-9-yl, 2-amino-purin-9-yl, 2-amino-6-chloro-purin-9-yl, 2-6-diamino-purin-9-yl, 3-carboxamido-1,2,4-triazol-1-yl, 3-deaza-adenin-9-yl, 3-deaza-guanin-9-yl, 3-deaza-inosin-9-yl, 3-deaza-2-amino-purin-9-yl, 3-deaza-2-amino-6-chloro-purin-9-yl, 3-deaza-2,6-diamino-purin-9-yl, 7-deaza-adenin-9-yl, 7-deaza-guanin-9-yl, 7-deaza-inosin-9-yl, 7-deaza-2-amino-purin-9-yl, 7-deaza-2-amino-6-chloro-purin-9-yl, 7-deaza-2-6-diamino-purin-9-yl, 7-deaza-8-aza-adenin-9-yl, 7-deaza-8-aza-guanin-9-yl, 7-deaza-8-aza-inosin-9-yl, 7-deaza-8-aza-2-amino-purin-9-yl, 7-deaza-8-aza-2-amino-6-chloro-purin-9-yl, 7-deaza-8-aza-2-6-diamino-purin-9-yl, 8-aza-adenin-9-yl, 8-aza-guanin-9-yl, 8-aza-inosin-9-yl, 8-aza-2-amino-purin-9-yl, 8-aza-2-amino-6-chloro-purin-9-yl, 8-aza-2-6-diamino-purin-9-yl, 6-thio-purin, 6-methylthiopurin, 5-aza-thymin-1-yl, 5-aza-cytosin-1-yl, 5-aza-uracil-1-yl, 6-aza-thymin-1-yl, 6-aza-cytosin-1-yl, 6-aza-uracil-1-yl, 2-thiouracil-1-yl, 4-thiouracil-1-yl, 2-thiocytosine-1-yl, uracil-5-yl, 2-thiouracil-5-yl, 4-thiouracil-5-yl, substituted pyridine derivatives such as 6-azauracil, and azacytosine. In general, attachment may be at different positions in the ring at nitrogen or carbon. These B ring systems may be substituted with halo, alkyl, substituted alkyl (F, Cl, Br, I, OH), NH₂, N₃, aryl, substituted aryl (F, Cl, Br, I, OH, NH₂), aralkyl.

To further illustrate, some of the purine derivatives, the following structure is taken as B

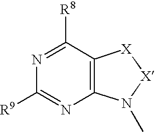

X and X′ is independently CH, N;

R⁸ and R⁹ independately is H, NH₂, OH, SH, Cl, Br, I, aryl, heterocycle, alkyl, alkene, alkyne, S-alkyl, S-aryl, S(O)-alkyl, SO₂-alkyl, SO₂NH₂, SO₂NH-alkyl, SO₂NH-aryl, NH-alkyl, NH-aryl, N(alkyl)₂, N(aryl)₂, O-alkyl, O-aryl, O-heterocycle,

and pharmaceutically acceptable salts thereof and prodrugs thereof.

Exceptions to the above compounds are when A is CH₂OH, CH₂F, CH₂N₃, CH₃ or —CH═CH₂ then B cannot be adenin-9-yl, 1-deazaadenin-9-yl, 7-deaza-8-azaadenin-9-yl, 8-azaadenin-9-yl, guanin-9-yl, 2,6-diaminopurin-9-yl, 2-aminopurin-9-yl, thymin-1-yl, cytosine-1-yl, 5-fluorocytosin-1-yl, 6-azacytosin-1 yl, 5-methylcytosin-1-yl, 5-bromovinyluracil-1-yl, 5-fluorouracil-1-yl or 5-trifluoromethyluracil-1-yl.

3. Compounds resented by the formula:

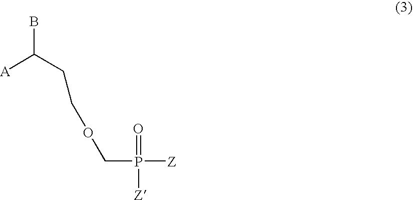

Wherein A is —CH₂)ₙ—R, —CH═CH₂, —CH₂—CH═CH₂, —O—(CH₂)ₙ—R, —CH(OH)CH₃, —CH(OH)—CH₂OH, —CH₂—CH(OH)CH₃, —CH₂—CH(OH)—CH₂OH, —CH(OH)—CH(OH)—CH₃;

R is H, OH, F, N₃, NH₂, CO₂H, SH, alkyl, S-alkyl, O-acyl, CONH₂, CONH-alkyl;

Z and Z′ independently is OR¹, OR², O—(CH₂)ₙ—O-alkyl or aminoacids and esters thereof

R¹ and R² independently is H, alkyl, aryl, pivaloyloxymethyl, phthalyl or substituted phthalyl, C(R³)₂OC(O)X(R⁴)a,

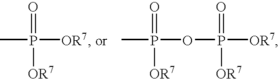

R³ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, or —OR⁵;

R⁴ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, —N(R⁶)₂ or —OR⁵;

R⁵ is C₁-C₁₂ alkyl, C₂-C₁₂ alkenyl, C₂-C₁₂ alkynyl or C₅-C₁₂ aryl;

provided that at least one R⁴ is not H; and a is 1 when X is CH₂, or direct bond, or 1 or 2 when X is N with the proviso that when a is 2 and X is N, (a) two N-linked R groups can be taken together to form a carbocyclic or oxygen containing heterocycle, (b) one N-linked R⁴ additionally can be —OR⁵ or (c) both N-linked R⁴ groups can be —H;

R⁶ is H or C₁-C₈ alkyl;

R⁷ is selected from H, alkyl, alkenyl, alkynyl, aryl, acyloxyalkyl, and pivaloyloxyalkyl;

B is selected from the group consisting of adenine, guanine, cytosine, uracil, thymine, modified purines and pyrimidines such as inosin-9-yl, 2-amino-purin-9-yl, 2-amino-6-chloro-purin-9-yl, 2-6-diamino-purin-9-yl, 3-carboxamido-1,2,4-triazol-1-yl, 3-deaza-adenin-9-yl, 3-deaza-guanin-9-yl, 3-deaza-inosin-9-yl, 3-deaza-2-amino-purin-9-yl, 3-deaza-2-amino-6-chloro-purin-9-yl, 3-deaza-2,6-diamino-purin-9-yl, 7-deaza-adenin-9-yl, 7-deaza-guanin-9-yl, 7-deaza-inosin-9-yl, 7-deaza-2-amino-purin-9-yl, 7-deaza-2-amino-6-chloro-purin-9-yl, 7-deaza-2-6-diamino-purin-9-yl, 7-deaza-8-aza-adenin-9-yl, 7-deaza-8-aza-guanin-9-yl, 7-deaza-8-aza-inosin-9-yl, 7-deaza-8-aza-2-amino-purin-9-yl, 7-deaza-8-aza-2-amino-6-chloro-purin-9-yl, 7-deaza-8-aza-2-6-diamino-purin-9-yl, 8-aza-adenin-9-yl, 8-aza-guanin-9-yl, 8-aza-inosin-9-yl, 8-aza-2-amino-purin-9-yl, 8-aza-2-amino-6-chloro-purin-9-yl, 8-aza-2-6-diamino-purin-9-yl, 6-thio-purin, 6-methylthiopurin, 5-aza-thymin-1-yl, 5-aza-cytosin-1-yl, 5-aza-uracil-1-yl, 6-aza-thymin-1-yl, 6-aza-cytosin-1-yl, 6-aza-uracil-1-yl, 2-thiouracil-1-yl, 4-thiouracil-1-yl, 2-thiocytosine-1-yl, uracil-5-yl, 2-thiouracil-5-yl, 4-thiouracil-5-yl, substituted pyridine derivatives such as 6-azauracil, and azacytosine. In general, attachment may be at different positions in the ring at nitrogen or carbon. These B ring systems may be substituted with halo, alkyl, substituted alkyl (F, Cl, Br, I, OH), NH₂, N₃, aryl, substituted aryl (F, Cl, Br, I, OH, NH₂), aralkyl.

To further illustrate, some of the purine derivatives, the following structure is taken as B

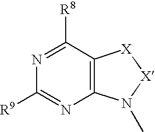

X and X′ is independently CH, N;

R⁸ and R⁹ independately is H, NH₂, OH, SH, Cl, Br, I, aryl, heterocycle, alkyl, alkene, alkyne, S-alkyl, S-aryl, S(O)-alkyl, SO₂-alkyl, SO₂NH₂, SO₂NH-alkyl, SO₂NH-aryl, NH-alkyl, NH-aryl, N(alkyl)₂, N(aryl)₂, O-alkyl, O-aryl, O-heterocycle,

and pharmaceutically acceptable salts thereof and prodrugs thereof.

Exceptions to the above compounds are when A is CH₂OH, CH₂F, CH₂N₃, CH₃ or —CH═CH₂ then B cannot be adenin-9-yl, 1-deazaadenin-9-yl, 7-deaza-8-azaadenin-9-yl, 8-azaadenin-9-yl, guanin-9-yl, 2,6-diaminopurin-9-yl, 2-aminopurin-9-yl, thymin-1-yl, cytosine-1-yl, 5-fluorocytosin-1-yl, 6-azacytosin-1 yl, 5-methylcytosin-1-yl, 5-bromovinyluracil-1-yl, 5-fluorouracil-1-yl or 5-trifluoromethyluracil-1-yl;

Compounds of the present invention are useful as therapeutic agents for RNA viral polymerase. Accordingly, another aspect of the present invention relates to pharmaceutical composition containing at least one of the above-disclosed compounds.

The present invention also relates to a method of inhibiting RNA polymerases in a patient by administering to the patient at least one of the above-disclosed compounds in an amount sufficient to inhibit viral RNA polymerases, such as HCV, HBV, small pox, Coxsackie A and B, Rhino, Echo, Ebola, polio and West Nile virus.

The present invention is also concerned with methods of using the compounds of the present invention in treating a patient suffering from RNA viral infections such as HCV, HBV, small pox, Ebola, polio, West Nile, Coxsackie A and B, Rhino, and Echo viral infection by administering to the patient an effective amount of at least one of the above-disclosed compounds.

Still other objects and advantages of the present invention will become readily apparent by those skilled in art from the following detailed description, wherein it is shown and described preferred embodiments of the invention, simply by way of illustration of the best mode contemplated of carrying out the invention. As will be realized, the invention is capable of other and different embodiments, and its several details are capable of modifications in various obvious aspects, without departing from the invention. Accordingly, the description to be regarded as illustrative in nature and not as restrictive.

### BEST AND VARIOUS MODES FOR CARRYING OUT INVENTION

Compounds of the present invention are represented by the formula:

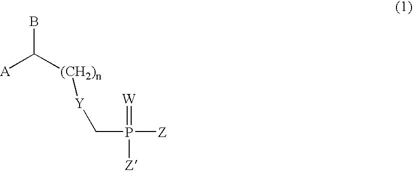

Wherein A is —(CH₂)ₙ—R, —CH═CH₂, —CH₂—CH═CH₂, —O—(CH₂)ₙ—R, —CH(OH)CH₃, —CH(OH)—CH₂OH, —CH₂—CH(OH)CH₃, —CH₂—CH(OH)—CH₂OH, —CH(OH)—CH(OH)—CH₃;

R is H, OH, F, N₃, NH₂, CO₂H, SH, alkyl, S-alkyl, O-acyl, CONH₂, CONH-alkyl;

Z and Z′ independently is OR¹, OR², O—(CH₂)ₙ—O-alkyl or aminoacids and esters thereof;

R¹ and R² independently is H, alkyl, aryl, pivaloyloxymethyl, phthalyl or substituted phthalyl, C(R³)₂OC(O)X(R⁴)a,

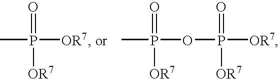

R³ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, or —OR⁵;

R⁴ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, —N(R⁶)₂ or —OR⁵;

R⁵ is C₁-C₁₂ alkyl, C₂-C₁₂ alkenyl, C₂-C₁₂ alkynyl or C₅-C₁₂ aryl;

provided that at least one R⁴ is not H; and a is 1 when X is CH₂, or direct bond, or 1 or 2 when X is N with the proviso that when a is 2 and X is N, (a) two N-linked R groups can be taken together to form a carbocyclic or oxygen containing heterocycle, (b) one N-linked R⁴ additionally can be —OR⁵ or (c) both N-linked R⁴ groups can be —H;

R⁶ is H or C₁-C₈ alkyl;

R⁷ is selected from H, alkyl, alkenyl, alkynyl, aryl, acyloxyalkyl, and pivaloyloxyalkyl;

n is 1-3;

Y is O, S or NH;

W is O or S;

B is selected from the group consisting of adenine, guanine, cytosine, uracil, thymine, modified purines and pyrimidines such as inosin-9-yl, 2-amino-purin-9-yl, 2-amino-6-chloro-purin-9-yl, 2-6-diamino-purin-9-yl, 3-carboxamido-1,2,4-triazol-1-yl, 3-deaza-adenin-9-yl, 3-deaza-guanin-9-yl, 3-deaza-inosin-9-yl, 3-deaza-2-amino-purin-9-yl, 3-deaza-2-amino-6-chloro-purin-9-yl, 3-deaza-2,6-diamino-purin-9-yl, 7-deaza-adenin-9-yl, 7-deaza-guanin-9-yl, 7-deaza-inosin-9-yl, 7-deaza-2-amino-purin-9-yl, 7-deaza-2-amino-6-chloro-purin-9-yl, 7-deaza-2-6-diamino-purin-9-yl, 7-deaza-8-aza-adenin-9-yl, 7-deaza-8-aza-guanin-9-yl, 7-deaza-8-aza-inosin-9-yl, 7-deaza-8-aza-2-amino-purin-9-yl, 7-deaza-8-aza-2-amino-6-chloro-purin-9-yl, 7-deaza-8-aza-2-6-diamino-purin-9-yl, 8-aza-adenin-9-yl, 8-aza-guanin-9-yl, 8-aza-inosin-9-yl, 8-aza-2-amino-purin-9-yl, 8-aza-2-amino-6-chloro-purin-9-yl, 8-aza-2-6-diamino-purin-9-yl, 6-thio-purin, 6-methylthiopurin, 5-aza-thymin-1-yl, 5-aza-cytosin-1-yl, 5-aza-uracil-1-yl, 6-aza-thymin-1-yl, 6-aza-cytosin-1-yl, 6-aza-uracil-1-yl, 2-thiouracil-1-yl, 4-thiouracil-1-yl, 2-thiocytosine-1-yl, uracil-5-yl, 2-thiouracil-5-yl, 4-thiouracil-5-yl, substituted pyridine derivatives such as 6-azauracil, and azacytosine. In general, attachment may be at different positions in the ring at nitrogen or carbon. These B ring systems may be substituted with halo, alkyl, substituted alkyl (F, Cl, Br, I, OH), NH₂, N₃, aryl, substituted aryl (F, Cl, Br, I, OH, NH₂), aralkyl.

To further illustrate, some of the purine derivatives, the following structure is taken as B

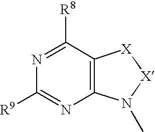

X and X′ is independently CH, N;

R⁸ and R⁹ independately is H, NH₂, OH, SH, Cl, Br, I, aryl, heterocycle, alkyl, alkene, alkyne, S-alkyl, S-aryl, S(O)-alkyl, SO₂-alkyl, SO₂NH₂, SO₂NH-alkyl, SO₂NH-aryl, NH-alkyl, NH-aryl, N(alkyl)₂, N(aryl)₂, O-alkyl, O-aryl, O-heterocycle,

and pharmaceutically acceptable salts thereof and prodrugs thereof.

Exceptions to the above compounds are when A is CH₂OH, CH₂F, CH₂N₃, CH₃ or —CH═CH₂ then B cannot be adenin-9-yl, 1-deazaadenin-9-yl, 7-deaza-8-azaadenin-9-yl, 8-azaadenin-9-yl, guanin-9-yl, 2,6-diaminopurin-9-yl, 2-aminopurin-9-yl, thymin-1-yl, cytosine-1-yl, 5-fluorocytosin-1-yl, 6-azacytosin-1 yl, 5-methylcytosin-1-yl, 5-bromovinyluracil-1-yl, 5-fluorouracil-1-yl or 5-trifluoromethyluracil-1-yl.

2) Compounds represented by the formula:

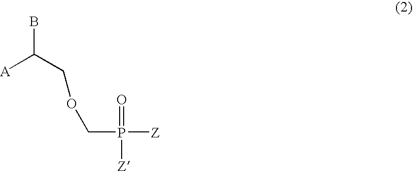

Wherein A is —(CH₂)ₙ—R, —CH═CH₂, —CH₂—CH═CH₂, —O—(CH₂)ₙ—R, —CH(OH)CH₃, —CH(OH)—CH₂OH, —CH₂—CH(OH)CH₃, —CH₂—CH(OH)—CH₂OH, —CH(OH)—CH(OH)—CH₃;

R is H, OH, F, N₃, NH₂, CO₂H, SH, alkyl, S-alkyl, O-acyl, CONH₂, CONH-alkyl;

Z and Z′ independently is OR¹, OR², O—(CH₂)ₙ—O-alkyl or aminoacids and esters thereof

R¹ and R² independently is H, alkyl, aryl, pivaloyloxymethyl, phthalyl or substituted phthalyl, C(R³)₂OC(O)X(R⁴)a,

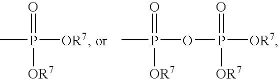

R³ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, or —OR⁵;

R⁴ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, —N(R⁶)₂ or —OR⁵;

R⁵ is C₁-C₁₂ alkyl, C₂-C₁₂ alkenyl, C₂-C₁₂ alkynyl or C₅-C₁₂ aryl;

provided that at least one R⁴ is not H; and a is 1 when X is CH₂, or direct bond, or 1 or 2 when X is N with the proviso that when a is 2 and X is N, (a) two N-linked R groups can be taken together to form a carbocyclic or oxygen containing heterocycle, (b) one N-linked R⁴ additionally can be —OR⁵ or (c) both N-linked R⁴ groups can be —H;

R⁶ is H or C₁-C₈ alkyl;

R⁷ is selected from H, alkyl, alkenyl, alkynyl, aryl, acyloxyalkyl, and pivaloyloxyalkyl;

B is selected from the group consisting of adenine, guanine, cytosine, uracil, thymine, modified purines and pyrimidines such as inosin-9-yl, 2-amino-purin-9-yl, 2-amino-6-chloro-purin-9-yl, 2-6-diamino-purin-9-yl, 3-carboxamido-1,2,4-triazol-1-yl, 3-deaza-adenin-9-yl, 3-deaza-guanin-9-yl, 3-deaza-inosin-9-yl, 3-deaza-2-amino-purin-9-yl, 3-deaza-2-amino-6-chloro-purin-9-yl, 3-deaza-2,6-diamino-purin-9-yl, 7-deaza-adenin-9-yl, 7-deaza-guanin-9-yl, 7-deaza-inosin-9-yl, 7-deaza-2-amino-purin-9-yl, 7-deaza-2-amino-6-chloro-purin-9-yl, 7-deaza-2-6-diamino-purin-9-yl, 7-deaza-8-aza-adenin-9-yl, 7-deaza-8-aza-guanin-9-yl, 7-deaza-8-aza-inosin-9-yl, 7-deaza-8-aza-2-amino-purin-9-yl, 7-deaza-8-aza-2-amino-6-chloro-purin-9-yl, 7-deaza-8-aza-2-6-diamino-purin-9-yl, 8-aza-adenin-9-yl, 8-aza-guanin-9-yl, 8-aza-inosin-9-yl, 8-aza-2-amino-purin-9-yl, 8-aza-2-amino-6-chloro-purin-9-yl, 8-aza-2-6-diamino-purin-9-yl, 6-thio-purin, 6-methylthiopurin, 5-aza-thymin-1-yl, 5-aza-cytosin-1-yl, 5-aza-uracil-1-yl, 6-aza-thymin-1-yl, 6-aza-cytosin-1-yl, 6-aza-uracil-1-yl, 2-thiouracil-1-yl, 4-thiouracil-1-yl, 2-thiocytosine-1-yl, uracil-5-yl, 2-thiouracil-5-yl, 4-thiouracil-5-yl, substituted pyridine derivatives such as 6-azauracil, and azacytosine. In general, attachment may be at different positions in the ring at nitrogen or carbon. These B ring systems may be substituted with halo, alkyl, substituted alkyl (F, Cl, Br, I, OH), NH₂, N₃, aryl, substituted aryl (F, Cl, Br, I, OH, NH₂), aralkyl.

To further illustrate, some of the purine derivatives, the following structure is taken as B

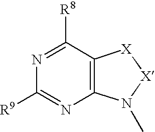

X and X′ is independently CH, N;

R⁸ and R⁹ independately is H, NH₂, OH, SH, Cl, Br, I, aryl, heterocycle, alkyl, alkene, alkyne, S-alkyl, S-aryl, S(O)-alkyl, SO₂-alkyl, SO₂NH₂, SO₂NH-alkyl, SO₂NH-aryl, NH-alkyl, NH-aryl, N(alkyl)₂, N(aryl)₂, O-alkyl, O-aryl, O-heterocycle,

and pharmaceutically acceptable salts thereof and prodrugs thereof.

Exceptions to the above compounds are when A is CH₂OH, CH₂F, CH₂N₃, CH₃ or —CH═CH₂ then B cannot be adenin-9-yl, 1-deazaadenin-9-yl, 7-deaza-8-azaadenin-9-yl, 8-azaadenin-9-yl, guanin-9-yl, 2,6-diaminopurin-9-yl, 2-aminopurin-9-yl, thymin-1-yl, cytosine-1-yl, 5-fluorocytosin-1-yl, 6-azacytosin-1 yl, 5-methylcytosin-1-yl, 5-bromovinyluracil-1-yl, 5-fluorouracil-1-yl or 5-trifluoromethyluracil-1-yl.

3. Compounds represented by the formula:

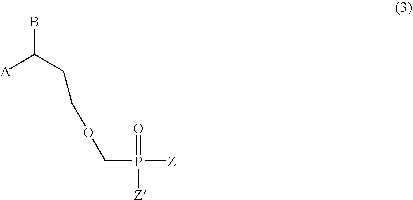

Wherein A is —(CH₂)ₙ—R, —CH═CH₂, —CH₂—CH═CH₂, —O—(CH₂)ₙ—R, —CH(OH)CH₃, —CH(OH)—CH₂OH, —CH₂—CH(OH)CH₃, —CH₂—CH(OH)—CH₂OH, —CH(OH)—CH(OH)—CH₃;

R is H, OH, F, N₃, NH₂, CO₂H, SH, alkyl, S-alkyl, O-acyl, CONH₂, CONH-alkyl;

Z and Z′ independently is OR¹, OR², O—(CH₂)ₙ—O-alkyl or aminoacids and esters thereof;

R¹ and R² independently is H, alkyl, aryl, pivaloyloxymethyl, phthalyl or substituted phthalyl, C(R³)₂OC(O)X(R⁴)a,

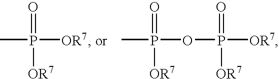

R³ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, or —OR⁵;

R⁴ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, —N(R⁶)₂ or —OR⁵;

R⁵ is C₁-C₁₂ alkyl, C₂-C₁₂ alkenyl, C₂-C₁₂ alkynyl or C₅-C₁₂ aryl;

provided that at least one R⁴ is not H; and a is 1 when X is CH₂, or direct bond, or 1 or 2 when X is N with the proviso that when a is 2 and X is N, (a) two N-linked R groups can be taken together to form a carbocyclic or oxygen containing heterocycle, (b) one N-linked R⁴ additionally can be —OR⁵ or (c) both N-linked R⁴ groups can be —H;

R⁶ is H or C₁-C₈ alkyl;

R⁷ is selected from H, alkyl, alkenyl, alkynyl, aryl, acyloxyalkyl, and pivaloyloxyalkyl;

B is selected from the group consisting of adenine, guanine, cytosine, uracil, thymine, modified purines and pyrimidines such as inosin-9-yl, 2-amino-purin-9-yl, 2-amino-6-chloro-purin-9-yl, 2-6-diamino-purin-9-yl, 3-carboxamido-1,2,4-triazol-1-yl, 3-deaza-adenin-9-yl, 3-deaza-guanin-9-yl, 3-deaza-inosin-9-yl, 3-deaza-2-amino-purin-9-yl, 3-deaza-2-amino-6-chloro-purin-9-yl, 3-deaza-2,6-diamino-purin-9-yl, 7-deaza-adenin-9-yl, 7-deaza-guanin-9-yl, 7-deaza-inosin-9-yl, 7-deaza-2-amino-purin-9-yl, 7-deaza-2-amino-6-chloro-purin-9-yl, 7-deaza-2-6-diamino-purin-9-yl, 7-deaza-8-aza-adenin-9-yl, 7-deaza-8-aza-guanin-9-yl, 7-deaza-8-aza-inosin-9-yl, 7-deaza-8-aza-2-amino-purin-9-yl, 7-deaza-8-aza-2-amino-6-chloro-purin-9-yl, 7-deaza-8-aza-2-6-diamino-purin-9-yl, 8-aza-adenin-9-yl, 8-aza-guanin-9-yl, 8-aza-inosin-9-yl, 8-aza-2-amino-purin-9-yl, 8-aza-2-amino-6-chloro-purin-9-yl, 8-aza-2-6-diamino-purin-9-yl, 6-thio-purin, 6-methylthiopurin, 5-aza-thymin-1-yl, 5-aza-cytosin-1-yl, 5-aza-uracil-1-yl, 6-aza-thymin-1-yl, 6-aza-cytosin-1-yl, 6-aza-uracil-1-yl, 2-thiouracil-1-yl, 4-thiouracil-1-yl, 2-thiocytosine-1-yl, uracil-5-yl, 2-thiouracil-5-yl, 4-thiouracil-5-yl, substituted pyridine derivatives such as 6-azauracil, and azacytosine. In general, attachment may be at different positions in the ring at nitrogen or carbon. These B ring systems may be substituted with halo, alkyl, substituted alkyl (F, Cl, Br, I, OH), NH₂, N₃, aryl, substituted aryl (F, Cl, Br, I, OH, NH₂), aralkyl.

To further illustrate, some of the purine derivatives, the following structure is taken as B

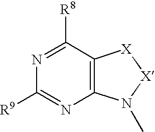

X and X′ is independently CH, N;

R⁸ and R⁹ independately is H, NH₂, OH, SH, Cl, Br, I, aryl, heterocycle, alkyl, alkene, alkyne, S-alkyl, S-aryl, S(O)-alkyl, SO₂-alkyl, SO₂NH₂, SO₂NH-alkyl, SO₂NH-aryl, NH-alkyl, NH-aryl, N(alkyl)₂, N(aryl)₂, O-alkyl, O-aryl, O-heterocycle,

and pharmaceutically acceptable salts thereof and prodrugs thereof.

Exceptions to the above compounds are when A is CH₂OH, CH₂F, CH₂N₃, CH₃ or —CH═CH₂ then B cannot be adenin-9-yl, 1-deazaadenin-9-yl, 7-deaza-8-azaadenin-9-yl, 8-azaadenin-9-yl, guanin-9-yl, 2,6-diaminopurin-9-yl, 2-aminopurin-9-yl, thymin-1-yl, cytosine-1-yl, 5-fluorocytosin-1-yl, 6-azacytosin-1 yl, 5-methylcytosin-1-yl, 5-bromovinyluracil-1-yl, 5-fluorouracil-1-yl or 5-trifluoromethyluracil-1-yl.

Definition of Terms

Listed below are definitions of various terms used to describe this invention. These definitions apply to the terms as they are used throughout this specification, unless otherwise limited in specific instances, either individually or as part of a larger group.

The terms “alkenyl” and “alkynyl” refer to straight or branched chain unsubstituted hydrocarbon groups typically having 2 to 8 carbon atoms.

The terms “substituted alkyl”, “substituted alkenyl” or substituted alkynyl” refer to an alkyl, alkenyl or alkynyl group substituted by, for example, one to four substituents, such as halo, trifluoromethyl, trifluoromethoxy, hydroxy, alkoxy, cycloalkyloxy, heterocyclooxy, oxo, alkanoyl, aryloxy, alkanoyloxy, amino, alkylamino, arylamino, aralkylamino, cycloalkylamino, heterocycloamino, disubstituted amines in which the 2 amino substituents are selected from alkyl, aryl or aralkyl, alkanoylamine, aroylamino, aralkanoylamino, substituted alkanolamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido (e.g. SO₂NH₂), substituted sulfonamido, nitro, cyano, carboxy, carbamyl (e.g. CONH₂), substituted carbamyl (e.g. CONH alkyl, CONH aryl, CONH aralkyl or cases where there are two substituents on the nitrogen selected from alkyl, aryl or aralkyl), alkoxycarbonyl, aryl, substituted aryl, guanidino and heterocyclos, such as indolyl, imidazolyl, furyl, thienyl, thiazolyl, pyrrolidyl, pyridyl, pyrimidyl and the like. Where noted above where the substituent is further substituted it will be with halogen, alkyl, alkoxy, aryl or aralkyl.

The term “halogen” or “halo” refers to fluorine, chlorine, bromine and iodine.

The term “aryl” refers to monocyclic or bicyclic aromatic hydrocarbon groups having 6 to 12 carbon atoms in the ring portion, such as phenyl, naphthyl, biphenyl and diphenyl groups, each of which may be substituted.

The term “aralkyl” or “alkylaryl” refers to an aryl group bonded directly through an alkyl group, such as benzyl or phenethyl.

The term “substituted aryl” or “substituted alkylaryl” refers to an aryl group or alkylaryl group substituted by, for example, one to four substituents such as alkyl; substituted alkyl, halo, trifluoromethoxy, trifluoromethyl, hydroxy, alkoxy, azido, cycloalkyloxy, heterocyclooxy, alkanoyl, alkanoyloxy, amino, alkylamino, aralkylamino, hydroxyalkyl, aminoalkyl, azidoalkyl, alkenyl, alkynyl, allenyl, cycloalkylamino, heterocycloamino, dialkylamino, alkanoylamino, thiol, alkylthio, cycloalkylthio, heterocyclothio, ureido, nitro, cyano, carboxy, carboxyalkyl, carbamyl, alkoxycarbonyl, alkylthiono, arylthiono, alkysulfonyl, sulfonamido, aryloxy and the like. The substituent may be further substituted by halo, hydroxy, alkyl, alkoxy, aryl, substituted aryl, substituted alkyl or aralkyl. “Substituted benzyl” refers to a benzyl group substituted by, for example, any of the groups listed above for substituted aryl.

The term “cycloalkyl” refers to optionally substituted, saturated cyclic hydrocarbon ring systems, preferably containing 1 to 3 rings and 3 to 7 carbons per ring which may be further fused with an unsaturated C₃-C₇ carbocyclic ring. Exemplary groups include cyclopropyl, cyclobutyl, cyclopentyl, cyclohexyl, cycloheptyl, cyclooctyl, cyclodecyl, cyclododecyl and adamantyl. Exemplary substituents include one or more alkyl groups as described above, or one or more groups described above as alkyl substituents.

The term “cycloalkenyl” refers to optionally substituted, unsaturated cyclic hydrocarbon ring systems, preferably containing 1 to 3 rings and 3-7 carbons per ring. Exemplary groups include cyclopentenyl and cyclohexenyl.

The term “alkyl” refers to straight or branched chain unsubstituted hydrocarbon groups of 1 to 20 carbon atoms, preferably 1 to 8 carbon atoms. The expression “lower alkyl” refers to unsubstituted alkyl groups of 1 to 4 carbon atoms. Alkyl groups may be substituted with halo (Cl, F, Br, I), OH, etc.

The term “aryl” refers to monocyclic or bicyclic aromatic hydrocarbon groups having 6 to 12 carbon atoms in the ring portion, such as phenyl, naphthyl, biphenyl and diphenyl and diphenyl groups, each of which may be substituted.

The term “acyl” refers to the residual moiety of a carboxylic acid group without the OH group of the acid and includes alkyl and acyl carboxylic acids. The alkyl group typically contains about 1-20 carbon atoms and more typically about 1-8 carbon atoms. The acyl group typically contains 6-12 carbon atoms. Examples of suitable acyl groups include acetyl and benzoyl.

Within the above-described definitions, certain embodiments are preferred. Preferred alkyl groups are lower alkyl groups containing 1 to about 8 carbon atoms, and more preferably 1 to about 5 carbon atoms, and can be straight, branched-chain or cyclic saturated aliphatic hydrocarbon groups.

Examples of suitable alkyl groups include methyl, ethyl and propyl. Examples of branched alkyl groups include isopropyl and t-butyl. An example of a suitable aralkyl group is phenethyl. Examples of suitable cycloalkyl groups typically contain 3-8 carbon atoms and include cyclopropyl, cyclobutyl, cyclopentyl and cyclohexyl. The aromatic or aryl groups are preferably phenyl and alkyl substituted aromatic groups (aralkyl) such as phenyl C₁₋₃ alkyl and benzyl.

Within the above-described definitions, certain embodiments are preferred. Preferred alkyl groups are lower alkyl groups containing 1 to about 8 carbon, and more preferably 1 to about 5 carbon atoms, and can be straight, branched-chain or cyclic saturated aliphatic hydrocarbon groups.

Examples of suitable alkyl groups include methyl, ethyl and propyl. Examples of branched alkyl groups include isopropyl and t-butyl. An example of a suitable alkylaryl group is phenethyl. Examples of suitable cycloalkyl groups typically contain 3-8 carbon atoms and include cyclopropyl, cyclobutyl, cyclopentyl and cyclohexyl. The aromatic or aryl groups are preferably phenyl or alkyl substituted aromatic groups (aralkyl) such as phenyl C₁₋₃ alkyl such as benzyl.

The terms “heterocycle”, “heterocyclic” and “heterocyclo” refer to an optionally substituted, fully saturated or unsaturated, aromatic or nonaromatic cyclic group, for example, which is a 4 to 7 membered monocyclic, 7 to 11 membered bicyclic, or 10 to 15 membered tricyclic ring system, which has at least one heteroatom and at least one carbon atom in the ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized and the nitrogen heteroatoms may also optionally be quaternized. The heterocyclic group may be attached at any heteroatom or carbon atom.

Prodrug forms of the compounds bearing various nitrogen functions (amino, hydroxyamino, amide, etc.) may include the following types of derivatives where each R group individually may be hydrogen, substituted or unsubstituted alkyl, aryl, alkenyl, alkynyl, heterocycle, alkylaryl, aralkyl, aralkenyl, aralkynl, cycloalkyl or cycloalkenyl groups as defined earlier.

(a) Carboxamides, —NHC(O)R

(b) Carbamates, —NHC(O)OR

(c) (Acyloxy)alkyl Carbamates, NHC(O)OROC(O)R

(d) Enamines, —NHCR(═CHCO₂R) or —NHCR(═CHCONR₂)

(e) Schiff Bases, —N═CR₂

(f) Mannich Bases (from carboximide compounds), RCONHCH₂NR₂

Preparations of such prodrug derivatives are discussed in various literature sources (examples are: Alexander et al., J. Med. Chem. 1988, 31, 318; Aligas-Martin et al., PCT WO pp/41531, p.30). The nitrogen function converted in preparing these derivatives is one (or more) of the nitrogen atoms of a compound of the invention.

Prodrug forms of carboxyl-bearing compounds of the invention include esters (—CO₂R) where the R group corresponds to any alcohol whose release in the body through enzymatic or hydrolytic processes would be at pharmaceutically acceptable levels. Another prodrug derived from a carboxylic acid form of the invention may be a quaternary salt type

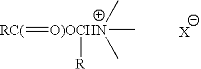

of structure described by Bodor et al., J. Med. Chem. 1980, 23, 469.

It is of course understood that the compounds of the present invention relate to all optical isomers and stereo-isomers at the various possible atoms of the molecule.

Pharmaceutically acceptable salts of the compounds of the present invention include those derived from pharmaceutically acceptable inorganic or organic acids. Examples of suitable acids include hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, formic, benzoic, malonic, naphthalene-2-sulfonic, trifluoroacetic and benzenesulfonic acids. Salts derived from appropriate bases include alkali such as sodium and ammonia.

The compounds of the present invention can be synthesized by persons skilled in the art once aware of the present disclosure without undue experimentation. Procedures are available in the chemical literature suitable for preparing the requisite sugars or nucleosides. Along these lines, see Choi, Jong-Ryoo; Kim, Jeong-Min; Roh, Kee-Yoon; Cho, Dong-Gyu; Kim, Jae-Hong; Hwang, Jae-Taeg; Cho, Woo-Young; Jang, Hyun-Sook; Lee, Chang-Ho; Choi, Tae-Saeng; Kim, Chung-Mi; Kim, Yong-Zu; Kim, Tae-Kyun; Cho, Seung-Joo; Kim, Gyoung-Won PCT Int. Appl. (2002), 100 pp. WO 0257288 A1 20020725. Holy, Antonin; Votruba, Ivan; Tloustova, Eva; Masojidkova, Milena. Collection of Czechoslovak Chemical Communications (2001), 66(10), 1545-1592. Rejman, Dominik; Masojidkova, Milena; De Clercq, Eric; Rosenberg, Ivan Nucleosides, Nucleotides & Nucleic Acids (2001), 20(8), 1497-1522; Ubasawa, Masaru; Sekiya, Kouichi PCT Int. Appl. (2001), 39 pp WO 0164693 A1 20010907. Otmar, Miroslav; Masojfdkova, Milena; Votruba, Ivan; Holy, Antonin. Collection of Czechoslovak Chemical Communications (2001), 66(3), 500-506. Michal; Hocek, Michal; Holy, Antonin. Collection of Czechoslovak Chemical Communications (2000), 65(8), 1357-1373. Jeffery, A. L.; Kim, J.-H.; Wiemer, D. F. Tetrahedron (2000), 56(29), 5077-5083. Holy, Antonin; Guenter, Jaroslav; Dvorakova, Hana; Masojidkova, Milena; Andrei, Graciela; Snoeck, Robert; Balzarini, Jan; De Clercq, Erik. Journal of Medicinal Chemistry (1999), 42(12), 2064-2086. Janeba, Zlatko; Holy, Antonin; Masojidkova, Milena. Collection of Czechoslovak Chemical Communications (2001), 66(9), 1393-1406. Holy, Antonin; Guenter, Jaroslav; Dvorakova, Hana; Masojidkova, Milena; Andrei, Graciela; Snoeck, Robert; Balzarini, Jan; De Clercq, Erik. Journal of Medicinal Chemistry (1999), 42(12), 2064-2086. Dang, Qun; Erion, Mark D.; Reddy, M. Rami; Robinsion, Edward D.; Kasibhatla, Srinivas Rao; Reddy, K. Raja. PCT Int. Appl. (1998), 126 pp WO 9839344 A1 19980911. Arimilli, Murty N.; Cundy, Kenneth C.; Dougherty, Joseph P.; Kim, Choung U.; Oliyai, Reza; Stella, Valentino J. PCT Int. Appl. (1998), 74 pp WO 9804569. Sekiya, Kouichi; Takashima, Hideaki; Ueda, Naoko; Kamiya, Naohiro; Yuasa, Satoshi; Fujimura, Yoshiyuki; Ubasawa, MasaruJournal of Medicinal Chemistry (2002), 45(14), 3138-3142. Ubasawa, Masaru; Sekiya, Kouichi; Takashima, Hideaki; Ueda, Naoko; Yuasa, Satoshi; Kamiya, Naohiro. Eur. Pat. Appl. (1997),56 pp EP 785208 A1 19970723. Hocek, Michal; Masojidkova, Milena; Holy, Antonin, Collection of Czechoslovak Chemical Communications (1997), 62(1), 136-146. Holy, Antonin; Votruba, Ivan; Tloustova, Eva; Masojidkova, Milena. Collection of Czechoslovak Chemical Communications (2001), 66(10), 1545-1592. Holy, Antonin; De Clercq, Erik Desire Alice. PCT Int. Appl. (1996), 57 pp. WO 9633200 A1 19961024. Rejman, Dominik; Rosenberg, Ivan. Collection of Czechoslovak Chemical Communications (1996), 61(Spec. Issue), S122-S123. Holy, Antonin; Dvorakova, Hana; Jindrich, Jindrich; Masojidkova, Milena; Budesinsky, Milos; Balzarini, Jan; Andrei, Graciella; De Clercq, Erik. Journal of Medicinal Chemistry (1996), 39(20), 4073-4088. Guanti, Giuseppe; Merlo, Valeria; Narisano, Enrica. Tetrahedron (1995), 51(35), 9737-46. Takashima, Hideaki; Inoue, Naoko; Ubasawa, Masaru; Sekiya, Kouichi; Yabuuchi, Shingo Eur. Pat. Appl. (1995), 88 pp. EP 632048 A1 19950104. Alexander, Petr; Holy, Antonin; Masojidkova, Milena, Collection of Czechoslovak Chemical Communications (1994), 59(8), 1853-69. Alexander, Petr; Holy, Antonin; Masojidkova, Milena; Collection of Czechoslovak Chemical Communications (1994), 59(8), 1853-69. Jindrich, Jindrich; Holy, Antonin; Dvorakova, Hana. Collection of Czechoslovak Chemical Communications (1993), 58(7), 1645-67. Holy, Antonin. Collection of Czechoslovak Chemical Communications (1993), 58(3), 649-74. Guanti, Giuseppe; Merlo, Valeria; Narisano, Enrica; Tetrahedron (1995), 51(35), 9737-46. Emishetti, Purushotham; Brodfuehrer, Paul R.; Howell, Henry G.; Sapino, Chester, Jr. PCT Int. Appl. (1992),43 pp. WO 9202511 A1 19920220. Glazier, Arnold. PCT Int. Appl. (1991), 131 pp. WO 9119721. Kim, Choung Un; Luh, Bing Yu; Misco, Peter F.; Bronson, Joanne J.; Hitchcock, Michael J. M.; Ghazzouli, Ismail; Martin, John C Journal of Medicinal Chemistry (1990), 33(4), 1207-13. Rosenberg, Ivan; Holy, Antonin; Masojidkova, Milena. Collection of Czechoslovak Chemical Communications (1988), 53(11B), 2753-77. Rosenberg, Ivan; Holy, Antonin; Masojidkova, Milena. Collection of Czechoslovak Chemical Communications (1988), 53(11B), 2753-77.

However, the following schemes illustrate methods for preparing compounds of the present invention. In order to facilitate an understanding of the present invention, the general methods will be discussed with respect to preparing various preferred compounds of the present invention.

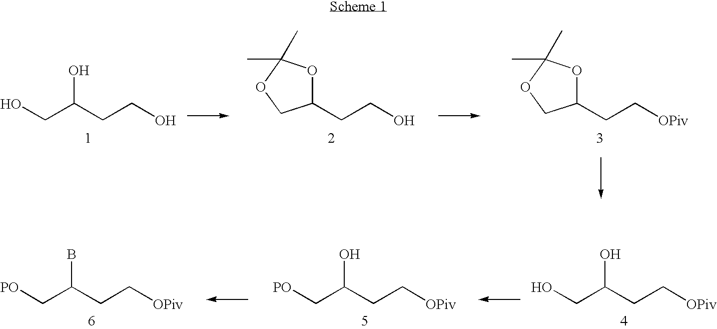

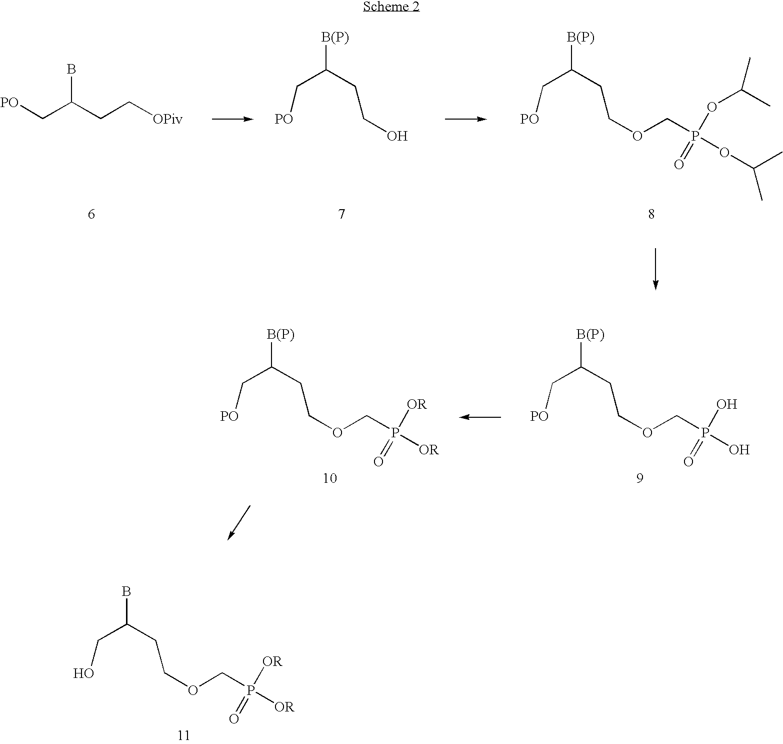

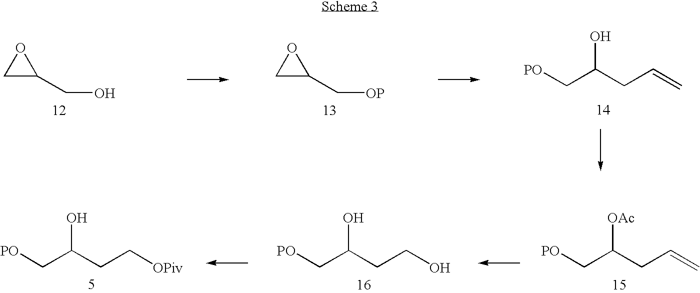

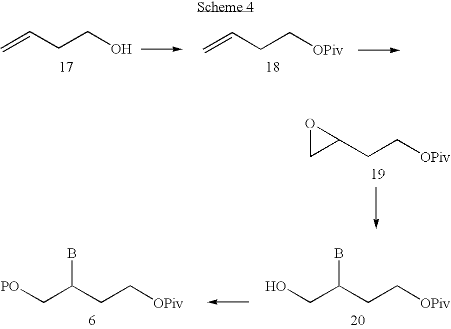

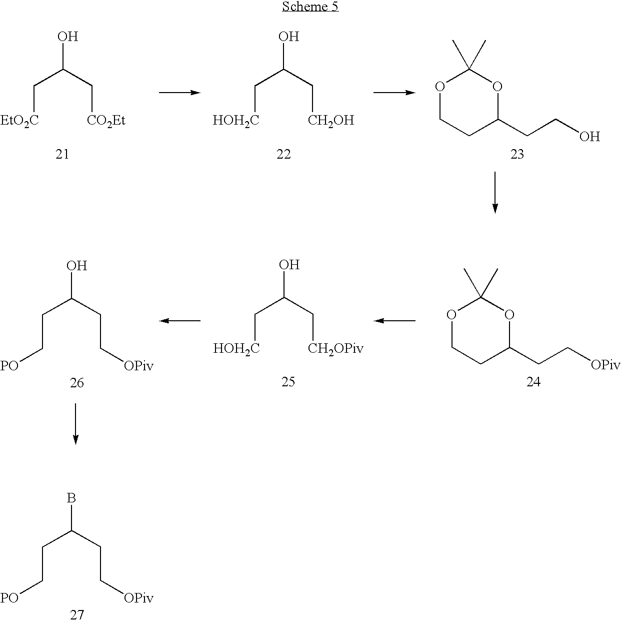

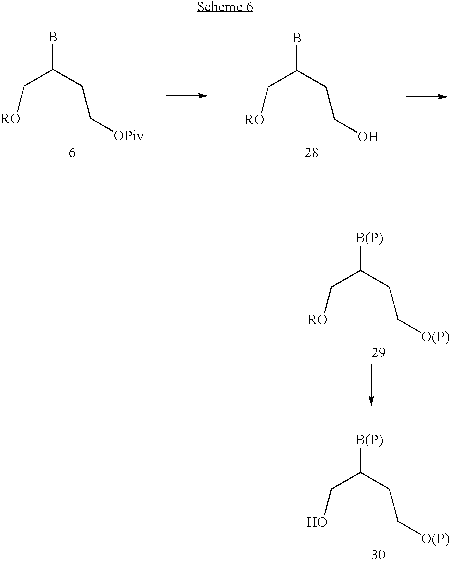

The following are the general methods for the preparation of the compounds claimed:

For the protection and deprotection of hydroxyl; 1,2-diols; carbonyl; carboxyl; thiol, amino, and phosphate groups, the standard procedures reported in “Protective Groups in Organic Synthesis by Theodora W. Greene and Peter G. M. Wuts” have been used. The particular group given in the synthesis can be replaced by any other suitable protecting group reported in the literature. The bases as described under ‘B’ substituents are either commercially available or can be prepared by the literature methods. The base can be suitably protected at amino or hydroxyl groups as needed by the standard methods of protection.

Scheme-1:

1,2,4-Butanetriol (1, purchased commercially or prepared by reduction of malic acid or its esters) is converted to 1,2-protected alcohol 2, such as isopropylidene, through standard procedures. The remaining hydroxyl group is again protected by a base sensitive group, such as pivaloyl to give 3. The isopropropylidene group in 3 is removed under acidic conditions to give 4 and the primary hydroxyl group is protected with a suitable protecting group (P) such as, trityl, monomethoxytrityl, tert-butyldimethylsilyl or tert-butyldiphenylsilyl group to give 5. The secondary hydroxyl group of 5 is converted to 6 through, i) Mitsunobu reaction using triphenylphosphine, diethyl or diisopropyl azodicarboxylate and a suitably protected or unprotected base, such as adenine, modified adenine, 6-chloropurine, cytosine, modified cytosine, uracil, modified uracil, protected guanine, modified guanine, etc. Any suitable base as given under ‘B’ substituents can be used before or after suitable protection, or ii) conversion of free hydroxyl group into a suitable leaving group such as, tosyl, methanesulphonyl, trifluoromethanesulphonyl, bromo or iodo and reacting with a protected or unprotected base under standard conditions as given in the literature.

Scheme-2:

If hydroxyl or amino groups of the base in compound 6 are not protected, those groups are protected with a acid sensitive and base stable groups, such as trityl or monomethoxytrityl and the pivaloyl group is removed under basic conditions to give 7. Further reaction of 7 with a suitable phosphorylating reagent [TsO—CH₂—P(O)(OEt)₂, TsO—CH₂—P(O)(O-iPr)₂, F₃CO₂SO—CH₂—P(O)(OEt)₂, F₃CO₂SO—CH₂—P(O)(O-iPr)₂] or any other reagent reported in the literature with a suitable base, such as sodium hydride or lithium tert-butoxide to give 8. The protecting group of phosphonate (ethyl or isopropyl) is removed according to the procedures given in the literature, preferably with trimethylsilyl bromide or trimethylsilyl iodide. When we have acid sensitive groups in the molecule, some base such as triethylamine can be used in the reaction to neutralize the acid generated in the reaction mixture. Treatment of compound 9 with a different prodrug synthon having a leaving group generate the prodrugs 10. The examples of various prodrugs are reported in the literature. Further deprotection gives the final prodrugs 11.

Scheme-3

Compound 5 is also prepared by an alternative route starting from glycidol 12, which is converted to the target using the procedures as described in schemes 1 and 2. The hydroxyl group of glycidol is protected by a suitable group such as trityl, monomethoxytrityl, tert-butyldimethylsilyl or tert-butyldiphenylsilyl to give 13, which is reacted with vinyl magnesium bromide to give 14. The acetylation results into protection of the hydroxyl group to give 15. Oxidation of the double bond in 15 is achieved by osmium tetraoxide, potassium permanganate or any other suitable oxidizing agent; 1,2 diol is cleaved using periodate; the resultant aldehyde is reduced with any reducing agent, such as sodium borohydride; and the resultant compound on treatment with a base yields compound 16. The primary hydroxyl group is protected with a base sensitive group, such as pivaloyl to give 5.

Scheme-4:

The alternative route for the preparation of 6 is illustrated in this scheme. The hydroxyl group of 3-buten-1-ol 17 is protected with a base sensitive group such as pivaloyl and the epoxide is formed by m-chloro perbenzoic acid reaction to give 19. The epoxide 19 is opened with a suitably protected base in the presence of an acid catalyst, such as trimethylsilyl triflate to give the desired compound 20. If another isomer is also formed, they are separated by chromatography. The protection of free hydroxyl with trityl, monomethoxytrityl, tert-butyldimethylsilyl or tert-butyldiphenylsilyl provides compound 6, which is then converted to the desired targets according to scheme 2.

Scheme-5:

Diethyl-3-hydroxyglutarate 21 is reduced to give triol 22, where 1,3-diol protection is achieved using the standard procedures given in “Protective Groups in Organic Synthesis” to give 23. The primary hydroxyl is protected with a base sensitive group, such as pivaloyl and acetonide is opened with acetic acid to give 25. Again the protection of primary hydroxyl is achieved using acid sensitive groups, such as trityl, monomethoxytrityl, tert-butyldimethylsilyl or tert-butyldiphenylsilyl and the resultant compound 26 is converted into 27 by the procedures given in scheme-1. Further conversions to the target molecules are the same as in scheme-2.

Scheme-6:

Compound 6, where the protecting group on the primary hydroxyl is tert-butyldimethylsilyl or tert-butyldiphenylsilyl, is reacted with a base to give 28 and the resultant hydroxyl and amino or hydroxyl groups of the base are protected with an acid sensitive moiety such as, trityl or monomethoxytrityl to give 29. Compound 29 on reaction with tetrabutyl ammonium fluoride produces 30, which is further converted to the targets according to the methods given in scheme-2.

All the methods of functional group transformations are described in the literature, particularly in “Comprehensive Organic Transformations by Richard C. Larock” and “Reagents for Organic Synthesis by Fieser and Fieser”. The same methods are used for the following transformations or preparations of the compounds.

Compounds of the structures 1, 2 and 3, when A=—(CH₂)nR where n=1 and R is H; F; N₃; NH₂; and CO₂H are prepared from the compound 30, where hydroxyl is transformed through deoxygenation; diethylaminosulfur trifluoride (DAST) reaction; Mitsunobu or through mesyl reaction with sodium azide; reduction of azide; and oxidation, respectively. The same way when n=2 or 3, the compounds are prepared from the corresponding alcohols. The further transformations are same as given in scheme-2.

Compounds of the structures 1, 2 and 3, when A=

—CH═CH₂ are prepared from the compound 30 by oxidation and Wittig reaction;

—CH₂CH═CH₂ are prepared from 7 by oxidation and Wittig reaction;

—CH(OH)CH₃ are prepared from 30 by oxidation and Grignard reaction;

—CH(OH)CH₂OH are prepared from the corresponding vinyl compound by dihydroxylation;

—CH₂CH(OH)CH₃ are prepared from 7 by oxidation and Grignard reaction;

—CH₂CH(OH)CH₂OH are prepared from the corresponding allyl compound by dihydroxylation.

The compounds of the structure 1, when Y=

—O are prepared by the methods given in schemes 1 to 6.

—CH₂ are prepared from the corresponding hydroxy compounds by converting into a bromo or iodo compound using standard procedures and following the same chemistry as described in scheme-2.

—S are prepared from the corresponding thiol, which are obtained from bromo derivatives by the reaction of thiourea or acetylthiourea.

—NH are prepared by converting alcohols to amino through azide and the reacting amino with phosphorus trichloride; further reaction with a prodrug derivative and finally oxidizing phosphorus to give the target prodrug molecules.

Examples of some specific compounds within the scope of the present invention are:

[3-(6-amino-9H-purin-9-yl)-4-hydroxybutoxy]methylphosphonic acid

[3-(2-amino-6-oxo-1,6-dihydro-9H-purin-9-yl)-4-hydroxybutoxy]methylphosphonic acid

[3-(2,4-dioxo-3,4-dihydro-1(2H)-pyrimidinyl)-4-hydroxybutoxy]methylphosphonic acid

[3-(4-amino-2-oxo-1(2H)-pyrimidinyl)-4-hydroxybutoxy]methylphosphonic acid

[2-(4-amino-2-oxo-1(2H)-pyrimidinyl)-4-hydroxybutoxy]methylphosphonic acid

[2-(2,4-dioxo-3,4-dihydro-1(2H)-pyrimidinyl)-4-hydroxybutoxy]methylphosphonic acid

[2-(6-amino-9H-purin-9-yl)-4-hydroxybutoxy]methylphosphonic acid

[2-(2-amino-6-oxo-1,6-dihydro-9H-purin-9-yl)-4-hydroxybutoxy]methylphosphonic acid

[4-hydroxy-2-(6-mercapto-9H-purin-9-yl)butoxy]methylphosphonic acid

{4-hydroxy-2-[6-(methylthio)-9H-purin-9-yl]butoxy}methylphosphonic acid

{2-[6-(dimethylamino)-9H-purin-9-yl]-4-hydroxybutoxy}methylphosphonic acid

{4-hydroxy-2-[6-(1H-pyrrol-1-yl)-9H-purin-9-yl]butoxy} methylphosphonic acid

{4-hydroxy-2-[6-(3-thienyl)-9H-purin-9-yl]butoxy}methylphosphonic acid

[4-hydroxy-2-(6-phenyl-9H-purin-9-yl)butoxy]methylphosphonic acid

[2-(6-chloro-9H-purin-9-yl)-4-hydroxybutoxy]methylphosphonic acid

[2-(6-bromo-9H-purin-9-yl)-4-hydroxybutoxy]methylphosphonic acid

(2-{6-[ethyl(methyl)amino]-9H-purin-9-yl}-4-hydroxybutoxy)methylphosphonic acid

{4-hydroxy-2-[6-(2-hydroxyethyl)-9H-purin-9-yl]butoxy}methylphosphonic acid

{4-hydroxy-2-[6-(hydroxymethyl)-9H-purin-9-yl]butoxy}methylphosphonic acid

[2-(4-amino-7H-pyrrolo[2,3-d]pyrimidin-7-yl)-4-hydroxybutoxy]methylphosphonic acid

[3-(4-amino-7H-pyrrolo[2,3-d]pyrimidin-7-yl)-4-hydroxybutoxy]methylphosphonic acid

[4-hydroxy-3-(6-mercapto-9H-purin-9-yl)butoxy]methylphosphonic acid

{4-hydroxy-3-[6-(methylthio)-9H-purin-9-yl]butoxy}methylphosphonic acid

{3-[6-(dimethylamino)-9H-purin-9-yl]-4-hydroxybutoxy}methylphosphonic acid

(3-{6-[ethyl(methyl)amino]-9H-purin-9-yl}-4-hydroxybutoxy)methylphosphonic acid

{4-hydroxy-3-[6-(1H-pyrrol-1-yl)-9H-purin-9-yl]butoxy} methylphosphonic acid

{4-hydroxy-3-[6-(3-thienyl)-9H-purin-9-yl]butoxy}methylphosphonic acid

[4-hydroxy-3-(6-phenyl-9H-purin-9-yl)butoxy]methylphosphonic acid

[3-(6-chloro-9H-purin-9-yl)-4-hydroxybutoxy]methylphosphonic acid

[3-(6-bromo-9H-purin-9-yl)-4-hydroxybutoxy]methylphosphonic acid

{4-hydroxy-3-[6-(hydroxymethyl)-9H-purin-9-yl]butoxy}methylphosphonic acid

{4-hydroxy-3-[6-(2-hydroxyethyl)-9H-purin-9-yl]butoxy}methylphosphonic acid

[3-(6-amino-9H-purin-9-yl)butoxy]methylphosphonic acid

[3-(6-amino-9H-purin-9-yl)-4-fluorobutoxy]methylphosphonic acid

[3-(6-amino-9H-purin-9-yl)-4-methoxybutoxy]methylphosphonic acid

[4-amino-3-(6-amino-9H-purin-9-yl)butoxy]methylphosphonic acid

[4-(acetyloxy)-3-(6-amino-9H-purin-9-yl)butoxy]methylphosphonic acid

[3-(6-amino-9H-purin-9-yl)-4-mercaptobutoxy]methylphosphonic acid

{[3-(6-amino-9H-purin-9-yl)-5-methoxy-5-oxopentyl]oxy}methylphosphonic acid

{[3-(6-amino-9H-purin-9-yl)-5-hydroxypentyl]oxy} methylphosphonic acid

[2-(6-amino-9H-purin-9-yl)-4-hydroxybutoxy]methylphosphonic acid

[2-(6-amino-9H-purin-9-yl)-4-methoxy-4-oxobutoxy]methylphosphonic acid

[2-(6-amino-9H-purin-9-yl)-4-mercaptobutoxy]methylphosphonic acid

[4-(acetyloxy)-2-(6-amino-9H-purin-9-yl)butoxy]methylphosphonic acid

[4-amino-2-(6-amino-9H-purin-9-yl)butoxy]methylphosphonic acid

[2-(6-amino-9H-purin-9-yl)-4-methoxybutoxy]methylphosphonic acid

[2-(6-amino-9H-purin-9-yl)-4-fluorobutoxy]methylphosphonic acid

Prodrugs [—CH₂—O—C(O)—C(CH₃)₃] of:

[3-(6-amino-9H-purin-9-yl)-4-hydroxybutoxy]methylphosphonic acid

[3-(2-amino-6-oxo-1,6-dihydro-9H-purin-9-yl)-4-hydroxybutoxy]methylphosphonic acid

[3-(2,4-dioxo-3,4-dihydro-1(2H)-pyrimidinyl)-4-hydroxybutoxy]methylphosphonic acid

[3-(4-amino-2-oxo-1(2H)-pyrimidinyl)-4-hydroxybutoxy]methylphosphonic acid

[2-(4-amino-2-oxo-1(2H)-pyrimidinyl)-4-hydroxybutoxy]methylphosphonic acid

[2-(2,4-dioxo-3,4-dihydro-1(2H)-pyrimidinyl)-4-hydroxybutoxy]methylphosphonic acid

[2-(6-amino-9H-purin-9-yl)-4-hydroxybutoxy]methylphosphonic acid

[2-(2-amino-6-oxo-1,6-dihydro-9H-purin-9-yl)-4-hydroxybutoxy]methylphosphonic acid

[4-hydroxy-2-(6-mercapto-9H-purin-9-yl)butoxy]methylphosphonic acid

{4-hydroxy-2-[6-(methylthio)-9H-purin-9-yl]butoxy}methylphosphonic acid

{2-[6-(dimethylamino)-9H-purin-9-yl]-4-hydroxybutoxy}methylphosphonic acid

{4-hydroxy-2-[6-(1H-pyrrol-1-yl)-9H-purin-9-yl]butoxy}methylphosphonic acid

{4-hydroxy-2-[6-(3-thienyl)-9H-purin-9-yl]butoxy}methylphosphonic acid

[4-hydroxy-2-(6-phenyl-9H-purin-9-yl)butoxy]methylphosphonic acid

[2-(6-chloro-9H-purin-9-yl)-4-hydroxybutoxy]methylphosphonic acid

[2-(6-bromo-9H-purin-9-yl)-4-hydroxybutoxy]methylphosphonic acid

(2-{6-[ethyl(methyl)amino]-9H-purin-9-yl}-4-hydroxybutoxy)methylphosphonic acid

{4-hydroxy-2-[6-(2-hydroxyethyl)-9H-purin-9-yl]butoxy} methylphosphonic acid

{4-hydroxy-2-[6-(hydroxymethyl)-9H-purin-9-yl]butoxy}methylphosphonic acid

[2-(4-amino-7H-pyrrolo[2,3-d]pyrimidin-7-yl)-4-hydroxybutoxy]methylphosphonic acid

[3-(4-amino-7H-pyrrolo[2,3-d]pyrimidin-7-yl)-4-hydroxybutoxy]methylphosphonic acid

[4-hydroxy-3-(6-mercapto-9H-purin-9-yl)butoxy]methylphosphonic acid

{4-hydroxy-3-[6-(methylthio)-9H-purin-9-yl]butoxy}methylphosphonic acid

{3-[6-(dimethylamino)-9H-purin-9-yl]-4-hydroxybutoxy} methylphosphonic acid

(3-{6-[ethyl(methyl)amino]-9H-purin-9-yl}-4-hydroxybutoxy)methylphosphonic acid

{4-hydroxy-3-[6-(1H-pyrrol-1-yl)-9H-purin-9-yl]butoxy}methylphosphonic acid

{4-hydroxy-3-[6-(3-thienyl)-9H-purin-9-yl]butoxy}methylphosphonic acid

[4-hydroxy-3-(6-phenyl-9H-purin-9-yl)butoxy]methylphosphonic acid

[3-(6-chloro-9H-purin-9-yl)-4-hydroxybutoxy]methylphosphonic acid

[3-(6-bromo-9H-purin-9-yl)-4-hydroxybutoxy]methylphosphonic acid

{4-hydroxy-3-[6-(hydroxymethyl)-9H-purin-9-yl]butoxy}methylphosphonic acid

{4-hydroxy-3-[6-(2-hydroxyethyl)-9H-purin-9-yl]butoxy}methylphosphonic acid

[3-(6-amino-9H-purin-9-yl)butoxy]methylphosphonic acid

[3-(6-amino-9H-purin-9-yl)-4-fluorobutoxy]methylphosphonic acid

[3-(6-amino-9H-purin-9-yl)-4-methoxybutoxy]methylphosphonic acid

[4-amino-3-(6-amino-9H-purin-9-yl)butoxy]methylphosphonic acid

[4-(acetyloxy)-3-(6-amino-9H-purin-9-yl)butoxy]methylphosphonic acid

[3-(6-amino-9H-purin-9-yl)-4-mercaptobutoxy]methylphosphonic acid

{[3-(6-amino-9H-purin-9-yl)-5-methoxy-5-oxopentyl]oxy}methylphosphonic acid

{[3-(6-amino-9H-purin-9-yl)-5-hydroxypentyl]oxy} methylphosphonic acid

[2-(6-amino-9H-purin-9-yl)-4-hydroxybutoxy]methylphosphonic acid

[2-(6-amino-9H-purin-9-yl)-4-methoxy-4-oxobutoxy]methylphosphonic acid

[2-(6-amino-9H-purin-9-yl)-4-mercaptobutoxy]methylphosphonic acid

[4-(acetyloxy)-2-(6-amino-9H-purin-9-yl)butoxy]methylphosphonic acid

[4-amino-2-(6-amino-9H-purin-9-yl)butoxy]methylphosphonic acid

[2-(6-amino-9H-purin-9-yl)-4-methoxybutoxy]methylphosphonic acid

[2-(6-amino-9H-purin-9-yl)-4-fluorobutoxy]methylphosphonic acid

Pursuant to the present invention, a study of the active site of HCV and other RNA polymerases as defined by x-ray crystallographic analysis indicates that many purine, pyrimidine and analogs thereof are tolerated in the part of the active site that binds the nucleic acid bases. It has also been determined according to the present invention that the part of the active site that binds the ribofuranose part of the nucleosides triphosphates can tolerate certain changes at the 2′ and 3′-hydroxyls of the ribofuranose ring. The amino groups can be substituted with alkyl and aralkyl groups. Therefore, the above disclosed compounds have been identified as inhibitors of RNA polymerase pursuant to this invention. Such inhibitors with sufficient potency will block the function of this enzyme preventing viral replication providing potential drugs for the treatment of diseases resulting from these viruses, such as hepatitis C, small pox, Ebola virus, West Nile virus, Polio, Coxsackie A and B, Rhino, and Echovirus.

HCV NS5B Polymerase Assays:

The activity of compounds predicted to inhibit HCV NS5B polymerase were examined in NS5B polymerase assay using two different RNA templates. Assays with poly(A) (primer dependent) or HCV RNA templates (primer independent, includes the genomic 3′-X stem loop) were adapted from literature and modified for the purpose of screening larger number of compounds. The reaction solution contained 0.1 M Hepes (pH 7.3), 1.75 mM MnCl₂, 4 mM DTT, 25 μg/mL rifampicin, 400 U/mL RNasin (Promega, Madison, Wis.), 0.6 μCi ³H-UTP or ³H-GTP (Amersham, Piscataway, N.J.), 60 μg/mL NS5B. For assays using homo-polymeric templates, primer and template (0.5 μg polyA/0.05 μg oligoU₁₆ or 0.5 μg polyC/0.05 μg oligoG₁₆) were pre-annealed at 95° C. for min followed by 37° C. for 10 min before their addition to the reaction. The total volume of the reaction mixture was 50 μL and incubations were at 30° C. for 2 h. Incorporation of tritium labeled RNA was determined by transferring the reaction solution to 0.8 ml of 0.1 mg/mL calf thymus DNA, the reaction products were precipitated with 0.45 mL cold 20% trichloroacetic acid solution on ice for 30 min. The labeled RNA products were collected on glass filters and washed extensively with 0.1 M acidic sodium pyrophosphate buffer and ethanol. The filter bound radioactivity was measured using a scintillation counter.

For the hetero-polymeric template, a segment of HCV RNA was labeled with biotin to allow high throughput screening of compounds. A PCR product encoding HCV genomic (+ strand) RNA from the 3′ non-coding region (nucleotides 9850 to 9970) was first amplified from a plasmid using a forward primer (GGATCCTAATACGACTCACTATAGGTGAAGATTGGGCTAACCACTCCAGG) containing a T7 promoter (underlined) and a reverse primer (GCCGGCCACATGATCTGCAGAGAG). This PCR product was used to prepare HCV RNA templates, which included the 3′-X stem loop. Biotinylated HCV RNA was produced from the PCR amplified template by in vitro transcription with biotin labeled nucleotides and T7 RNA polymerase. A 120 base biotinylated HCV (+) strand RNA product was purified with phenol chloroform extraction and size exclusion chromatography. Purified RNA was precipitated with ethanol and recovered by centrifugation. The assays using HCV RNA templates included 0.1 mM unlabeled nucleotide triphosphates and were done in 96-well streptavidin-coated Flash plates (NEN, Boston, Mass.). Biotinylated HCV RNA templates (0.2 μg per well) were pre-annealed prior to adding the reaction mixture. Assays were terminated by adding 150 μL of 20 mM EDTA (pH 8.0) in phosphate buffered saline to each well. Tritium counts were monitored using a Top counter (Packard Instrument, Meriden, Conn.). Different amounts of the test compound, typically ranging from 1 μM to 1 mM in a less than 5% of the total incubation volume, were added to measure NS5B polymerase inhibition. The same amount of solvent present in incubations containing inhibitors was added to control reactions. The IC₅₀ values were calculated by the following formula: % residual activity=100/(1+[I]/IC₅₀)^{s}, where [α]is the inhibitor concentration and “s” is the slope of the inhibition curve.

### Formulation

The compounds of the present invention can be administered by any conventional means available for use in conjunction with pharmaceuticals, either as individual therapeutic agents or in a combination of therapeutic agents. Example of these further therapeutic are interferon (IFN), interferon α-2a, interferon α-2b, consensus interferon (CIFN), ribavirin, amantadine, rimantadine, interleukine-12, ursodeoxycholic acid (UDCA), glycyrrhizin, and silybum marianum. They can be administered alone, but generally administered with a pharmaceutical carrier selected on the basis of the chosen route of administration and standard pharmaceutical practice.

The pharmaceutically acceptable carriers described herein, for example, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices.

The compounds of this invention can be administered by any conventional method available for use in conjunction with pharmaceuticals, either as individual therapeutic agents or in a combination of therapeutic agents.

The dosage administered well, of course, vary depending upon known factors, such as the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to 1000 milligrams (mg) per kilogram (kg) of body weight, with the preferred dose being 0.1 to about 30 mg/kg.

Dosage forms (compositions suitable for administration) contain from about 1 mg to about 500 mg of active ingredient per unit. In these pharmaceutical compositions, the active ingredient will ordinarily be present in an amount of about 0.5-95% weight based on the total weight of the composition.

The active ingredient can be administered orally in solid dosage forms, such as capsules, tablets, and powders, or in liquid dosage forms, such as elixirs, syrups and suspensions. It can also be administered parenterally, in sterile liquid dosage forms. The active ingredient can also be administered intranasally (nose drops) or by inhalation of a drug powder mist. Other dosage forms are potentially possible such as administration transdermally, via patch mechanism or ointment.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds of the present invention, alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present invention. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, Eds., 238-250(1982) and ASHP Handbook on Injectable Drugs, Toissel, 4th ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable pharmaceutical carriers are described in Remington's Pharmaceutical Sciences, Mack Publishing Company, a standard reference text in this field.

The dose administered to an animal, particularly a human, in the context of the present invention should be sufficient to effect a therapeutic response in the animal over a reasonable time frame. One skilled in the art will recognize that dosage will depend upon a variety of factors including a condition of the animal, the body weight of the animal.

A suitable dose is that which will result in a concentration of the active agent in a patient which is known to effect the desired response. The size of the dose also will be determined by the route, timing and frequency of administration as well as the existence, nature, and extend of any adverse side effects that might accompany the administration of the compound and the desired physiological effect.

Useful pharmaceutical dosage forms for administration of the compounds according to the present invention can be illustrated as follows:

### Hard Shell Capsules

A large number of unit capsules are prepared by filling standard two-piece hard gelatine capsules each with 100 mg of powdered active ingredient, 150 mg of lactose, 50 mg of cellulose and 6 mg of magnesium stearate.

### Soft Gelatin Capsules

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

### Tablets

A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

### Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Moreover, the compounds of the present invention can be administered in the form of nose drops, or metered dose and a nasal or buccal inhaler. The drug is delivered from a nasal solution as a fine mist or from a powder as an aerosol.

The foregoing description of the invention illustrates and describes the present invention. Additionally, the disclosure shows and describes only the preferred embodiments of the invention but, as mentioned above, it is to be understood that the invention is capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. Also, it is intended that the appended claims be construed to include alternative embodiments.

All publications, patents and patent applications cited in this specification are herein incorporated by reference, and for any and all purposes, as if each individual publication, patent or patent application were specifically and individually indicated to be incorporated by reference. In the case of inconsistencies, the present disclosure will prevail.

What is claimed is:

1. Compound represented by the formula: 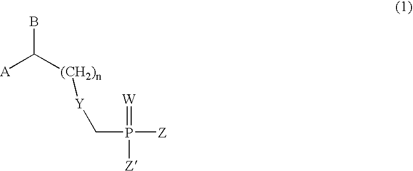
Wherein A is —(CH₂)ₙ—R, —CH═CH₂, —CH₂—CH═CH₂, —O—(CH₂)ₙ—R, —CH(OH)CH₃, —CH(OH)—CH₂OH, —CH₂—CH(OH)CH₃, —CH₂—CH(OH)—CH₂OH, or —CH(OH)—CH(OH)—CH₃;
R is H, OH, F, N₃, NH₂, CO₂H, SH, alkyl, S-alkyl, O-acyl, CONH₂, or CONH-alkyl;
Z and Z′ independently is OR¹, OR², O—(CH₂)ₙ—O-alkyl or aminoacids and esters thereof;
R¹ and R² independently is H, alkyl, aryl, pivaloyloxymethyl, phthalyl or substituted phthalyl, C(R³)₂OC(O)X(R⁴)a, 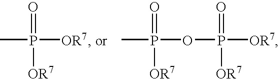
R³ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, or —OR⁵;
R⁴ is —H, C₁-C₁₂ alkyl, C₅-C₁₂ aryl, C₂-C₁₂ alkenyl, C₂-C₁₂ alkynyl, C₇-C₁₂ alkenylaryl, C₇-C₁₂ alkynylaryl, or C₆-C₁₂ alkaryl, any of which is unsubstituted or is substituted with 1 or 2 halo, cyano, azido, nitro, —N(R⁶)₂ or —OR⁵;
R⁵ is C₁-C₁₂ alkyl, C₂-C₁₂ alkenyl, C₂-C₁₂ alkynyl or C₅-C₁₂ aryl;
provided that at least one R⁴ is not H; and a is 1 when X is CH₂, or direct bond, or 1 or 2 when X is N with the proviso that when a is 2 and X is N, (a) two N-linked R groups can be taken together to form a carbocyclic or oxygen containing heterocycle, (b) one N-linked R⁴ additionally can be —OR⁵ or (c) both N-linked R⁴ groups can be —H;
R⁶ is H or C₁-C₈ alkyl;
R⁷ is selected from H, alkyl, alkenyl, alkynyl, aryl, acyloxyalkyl, and pivaloyloxyalkyl;
n is 1-3;
Y is O, S or NH;
W is O or S;
B is selected from the group consisting of adenine, guanine, cytosine, uracil, thymine, modified purines, modified pyrimidines and modified pyridines; and wherein B is optionally substituted with at least one member selected from the group consisting of halo, alkyl, substituted alkyl, NH₂, N₃, aryl, substituted aryl, aralkyl, wherein said substituted alkyl and said substituted aryl comprise at least one member selected from the group consisting of halo, OH and NH₂; and further provided that when A is CH₂OH, CH₂F, CH₂N₃, CH₃ or CH═CH₂ then B is other than adenin-9-yl, 1-deazaadenin-9-yl, 7-deaza-8-azaadenin-9-yl, 8-azaadenin-9-yl, guanin-9-yl, 2,6-diaminopurin-9-yl, 2-aminopurin-9-yl, thymin-1-yl, cytosine-1-yl, 5-fluorocytosin-1-yl, 6-azacytosin-1 yl, 5-methylcytosin-1-yl, 5-bromovinyluracil-1-yl, 5-fluorouracil-1-yl or 5-trifluoromethyluracil-1-yl; and pharmaceutically accepted salts thereof and prodrugs thereof.

2. The compound of claim 1 wherein said modified purines, pyrimidines and pyridines are selected from the group consisting of substituted and unsubstituted inosin-9-yl, 2-amino-purin-9-yl, 2-amino-6-chloro-purin-9-yl, 2-6-diamino-purin-9-yl, 3-carboxamido-1,2,4-triazol-1-yl, 3-deaza-adenin-9-yl, 3-deaza-guanin-9-yl, 3-deaza-inosin-9-yl, 3-deaza-2-amino-purin-9-yl, 3-deaza-2-amino-6-chloro-purin-9-yl, 3-deaza-2,6-diamino-purin-9-yl, 7-deaza-adenin-9-yl, 7-deaza-guanin-9-yl, 7-deaza-inosin-9-yl, 7-deaza-2-amino-purin-9-yl, 7-deaza-2-amino-6-chloro-purin-9-yl, 7-deaza-2-6-diamino-purin-9-yl, 7-deaza-8-aza-adenin-9-yl, 7-deaza-8-aza-guanin-9-yl, 7-deaza-8-aza-inosin-9-yl, 7-deaza-8-aza-2-amino-purin-9-yl, 7-deaza-8-aza-2-amino-6-chloro-purin-9-yl, 7-deaza-8-aza-2-6-diamino-purin-9-yl, 8-aza-adenin-9-yl, 8-aza-guanin-9-yl, 8-aza-inosin-9-yl, 8-aza-2-amino-purin-9-yl, 8-aza-2-amino-6-chloro-purin-9-yl, 8-aza-2-6-diamino-purin-9-yl, 6-thio-purin, 6-methylthiopurin, 5-aza-thymin-1-yl, 5-aza-cytosin-1-yl, 5-aza-uracil-1-yl, 6-aza-thymin-1-yl, 6-aza-cytosin-1-yl, 6-aza-uracil-1-yl, 2-thiouracil-1-yl, 4-thiouracil-1-yl, 2-thiocytosine-1-yl, uracil-5-yl, 2-thiouracil-5-yl, 4-thiouracil-5-yl, 6-azauracil, and azacytosine.

3. The compound of claim 1 wherein B is represented by the formula: 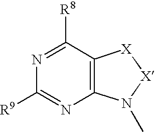
X and X′ is independently CH, N;
R⁸ and R⁹ independately is H, NH₂, OH, SH, Cl, Br, I, aryl, heterocycle, alkyl, alkene, alkyne, S-alkyl, S-aryl, S(O)-alkyl, SO₂-alkyl, SO₂NH₂, SO₂NH-alkyl, SO₂NH-aryl, NH-alkyl, NH-aryl, N(alkyl)₂, N(aryl)₂, O-alkyl, O-aryl, O-heterocycle.

4. The compound of claim 3 being represented by the formula:

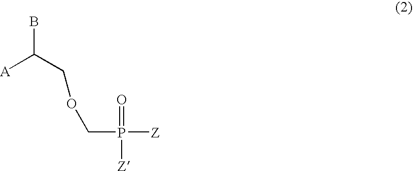

5. The compound of claim 1 being represented by the formula:

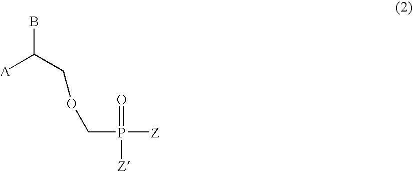

6. The compound of claim 3 being represented by the formula:

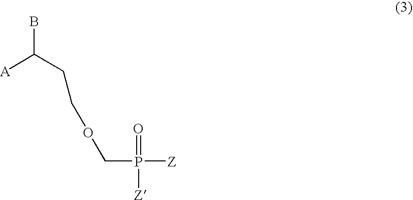

7. The compound of claim 1 being represented by the formula:

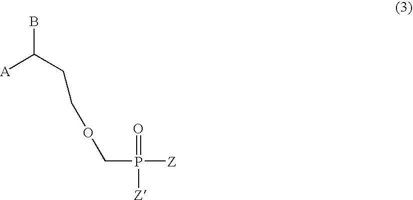

8. The compound of claim 1 being selected from the group consisting of
[3-(6-amino-9H-purin-9-yl)-4-hydroxybutoxy]methylphosphonic acid
[3-(2-amino-6-oxo-1,6-dihydro-9H-purin-9-yl)-4-hydroxybutoxy]methylphosphonic acid
[3-(2,4-dioxo-3,4-dihydro-1(2H)-pyrimidinyl)-4-hydroxybutoxy]methylphosphonic acid
[3-(4-amino-2-oxo-1(2H)-pyrimidinyl)-4-hydroxybutoxy]methylphosphonic acid
[2-(4-amino-2-oxo-1(2H)-pyrimidinyl)-4-hydroxybutoxy]methylphosphonic acid
[2-(2,4-dioxo-3,4-dihydro-1(2H)-pyrimidinyl)-4-hydroxybutoxy]methylphosphonic acid
[2-(6-amino-9H-purin-9-yl)-4-hydroxybutoxy]methylphosphonic acid
[2-(2-amino-6-oxo-1,6-dihydro-9H-purin-9-yl)-4-hydroxybutoxy]methylphosphonic acid
[4-hydroxy-2-(6-mercapto-9H-purin-9-yl)butoxy]methylphosphonic acid
{4-hydroxy-2-[6-(methylthio)-9H-purin-9-yl]butoxy}methylphosphonic acid
{2-[6-(dimethylamino)-9H-purin-9-yl]-4-hydroxybutoxy}methylphosphonic acid
{4-hydroxy-2-[6-(1H-pyrrol-1-yl)-9H-purin-9-yl]butoxy} methylphosphonic acid
{4-hydroxy-2-[6-(3-thienyl)-9H-purin-9-yl]butoxy} methylphosphonic acid
[4-hydroxy-2-(6-phenyl-9H-purin-9-yl)butoxy]methylphosphonic acid
[2-(6-chloro-9H-purin-9-yl)-4-hydroxybutoxy]methylphosphonic acid
[2-(6-bromo-9H-purin-9-yl)-4-hydroxybutoxy]methylphosphonic acid
(2-{6-[ethyl(methyl)amino]-9H-purin-9-yl}-4-hydroxybutoxy)methylphosphonic acid
{4-hydroxy-2-[6-(2-hydroxyethyl)-9H-purin-9-yl]butoxy}methylphosphonic acid
{4-hydroxy-2-[6-(hydroxymethyl)-9H-purin-9-yl]butoxy}methylphosphonic acid
[2-(4-amino-7H-pyrrolo[2,3-d]pyrimidin-7-yl)-4-hydroxybutoxy]methylphosphonic acid
[3-(4-amino-7H-pyrrolo[2,3-d]pyrimidin-7-yl)-4-hydroxybutoxy]methylphosphonic acid
[4-hydroxy-3-(6-mercapto-9H-purin-9-yl)butoxy]methylphosphonic acid
{4-hydroxy-3-[6-(methylthio)-9H-purin-9-yl]butoxy}methylphosphonic acid
{3-[6-(dimethylamino)-9H-purin-9-yl]-4-hydroxybutoxy}methylphosphonic acid
(3-{6-[ethyl(methyl)amino]-9H-purin-9-yl}-4-hydroxybutoxy)methylphosphonic acid
{4-hydroxy-3-[6-(1H-pyrrol-1-yl)-9H-purin-9-yl]butoxy} methylphosphonic acid
{4-hydroxy-3-[6-(3-thienyl)-9H-purin-9-yl]butoxy}methylphosphonic acid
[4-hydroxy-3-(6-phenyl-9H-purin-9-yl)butoxy]methylphosphonic acid
[3-(6-chloro-9H-purin-9-yl)-4-hydroxybutoxy]methylphosphonic acid
[3-(6-bromo-9H-purin-9-yl)-4-hydroxybutoxy]methylphosphonic acid
{4-hydroxy-3-[6-(hydroxymethyl)-9H-purin-9-yl]butoxy}methylphosphonic acid
{4-hydroxy-3-[6-(2-hydroxyethyl)-9H-purin-9-yl]butoxy}methylphosphonic acid
[3-(6-amino-9H-purin-9-yl)butoxy]methylphosphonic acid
[3-(6-amino-9H-purin-9-yl)-4-fluorobutoxy]methylphosphonic acid
[3-(6-amino-9H-purin-9-yl)-4-methoxybutoxy]methylphosphonic acid
[4-amino-3-(6-amino-9H-purin-9-yl)butoxy]methylphosphonic acid
[4-(acetyloxy)-3-(6-amino-9H-purin-9-yl)butoxy]methylphosphonic acid
[3-(6-amino-9H-purin-9-yl)-4-mercaptobutoxy]methylphosphonic acid
{[3-(6-amino-9H-purin-9-yl)-5-methoxy-5-oxopentyl]oxy}methylphosphonic acid
{[3-(6-amino-9H-purin-9-yl)-5-hydroxypentyl]oxy} methylphosphonic acid
[2-(6-amino-9H-purin-9-yl)-4-hydroxybutoxy]methylphosphonic acid
[2-(6-amino-9H-purin-9-yl)-4-methoxy-4-oxobutoxy]methylphosphonic acid
[2-(6-amino-9H-purin-9-yl)-4-mercaptobutoxy]methylphosphonic acid
[4-(acetyloxy)-2-(6-amino-9H-purin-9-yl)butoxy]methylphosphonic acid
[4-amino-2-(6-amino-9H-purin-9-yl)butoxy]methylphosphonic acid
[2-(6-amino-9H-purin-9-yl)-4-methoxybutoxy]methylphosphonic acid
[2-(6-amino-9H-purin-9-yl)-4-fluorobutoxy]methylphosphonic acid; and pivaloyl prodrugs thereof.

9. A pharmaceutical composition comprising a compound according to any one of claims 1-8.

10. The composition of claim 9 which further comprises a pharmaceutical carrier.

11. A method for inhibiting RNA viral polymerase in a patient by administering to the patient at least one compound according to any one of claims 1 to 8.

12. A method for inhibiting HCV polymerase in a patient by administering to the patient at least one compound according to any of claims 1 to 8.

13. A method for inhibiting HBV polymerase in a patient by administering to the patient at least one compound according to any of claims 1 to 8.

14. A method for inhibiting Rhino polymerase in a patient in need thereof by administering to the patient an effective amount of at least one compound according to any of claims 1 to 8.

15. A method for inhibiting small pox polymerase in a patient in need thereof by administering to the patient an effective amount of at least one compound according to any of claims 1 to 8.

16. A method for inhibiting Ebola polymerase in a patient in need thereof by administering to the patient an effective amount of at least one compound according to any of claims 1 to 8.

17. A method for inhibiting polio virus polymerase in a patient in need thereof by administering to the patient an effective amount of at least one compound according to any of claims 1 to 8.

18. A method for inhibiting West Nile polymerase in a patient in need thereof by administering to the patient an effective amount of at least one compound according to any of claims 1 to 8.

19. A method for inhibiting Coxsackie A polymerase in a patient in need thereof by administering to the patient an effective amount of at least one compound according to any of claims 1 to 8.

20. A method for inhibiting Coxsackie B polymerase in a patient in need thereof by administering to the patient an effective amount of at least one compound according to any of claims 1 to 8.

21. A method for inhibiting Echo polymerase in a patient in need thereof by administering to the patient an effective amount of at least one compound according to any of claims 1 to 8.

22. A method for treating a patient suffering from an RNA viral infection which comprises administering to said patient an effective amount of at least one compound according to any one of the claims 1 to 8.

23. A method for treating a patient suffering from HCV which comprises administering to said patient an effective amount of at least one compound according to any one of the claims 1 to 8.

24. A method for treating a patient suffering from HBV which comprises administering to said patient an effective amount of at least one compound according to any one of the claims 1 to 8.

25. A method for treating a patient suffering from a Rhino viral infection which comprises administering to said patient an effective amount of at least one compound according to any one of the claims 1 to 8.

26. A method for treating a patient suffering from a small pox viral infection which comprises administering to said patient an effective amount of at least one compound according to any one of the claims 1 to 8.

27. A method for treating a patient suffering from a Ebola viral infection which comprises administering to said patient an effective amount of at least one compound according to any one of the claims 1 to 8.

28. A method for treating a patient suffering from a polio viral infection which comprises administering to said patient an effective amount of at least one compound according to any one of the claims 1 to 8.

29. A method for treating a patient suffering from a West Nile viral infection which comprises administering to said patient an effective amount of at least one compound according to any one of the claims 1 to 8.

30. A method for treating a patient suffering from a Coxsackie A viral infection which comprises administering to said patient an effective amount of at least one compound according to any one of the claims 1 to 8.

31. A method for treating a patient suffering from a Coxsackie B viral infection which comprises administering to said patient an effective amount of at least one compound according to any one of the claims 1 to 8.

32. A method for treating a patient suffering from an Echo viral infection which comprises administering to said patient an effective amount of at least one compound according to any one of the claims 1 to 8.

33. A method for inhibiting in a patient in need thereof a RNA viral polymerase which comprises administering to said patient an effective amount of at least one compound according to claims 1 to 8 and and at least one further therapeutic agent related from the group consisting of interferon (IFN), interferon α-2a, interferon α-2b, consensus interferon (CIFN), ribavirin, amantadine, rimantadine, interleukine-12, ursodeoxycholic acid (UDCA), glycyrrhizin, and silybum marianum.

34. The method of claim 33 wherein the RNA viral polymerase comprises at least one member selected from the group consisting of HCV polymerase, HBV polymerase, Rhino polymerase, small pox virus polymerase, Ebola virus polymerase, Coxsackie A and B polymerase, Echo polymerase and west Nile virus polymerase.

35. A method for treating a patient suffering from a RNA viral infection which comprises administering to the patient an effective amount of at least one compound according to claims 1 to 8 and at least one further therapeutic agent chosen from interferon (IFN), interferon α-2a, interferon α-2b, consensus interferon (CIFN), ribavirin, amantadine, rimantadine, interleukine-12, ursodeoxycholic acid (UDCA), glycyrrhizin, and silybum marianum.

36. The method of claim 35 wherein the RNA viral infection comprises at least one member selected from the group consisting of HCV, HBV, Coxsackie A, Coxsackie B, Echo, Rhino viral infection, small pox viral infection, Ebola viral infection, polio viral infection and West Nile viral infection.
